(19) United States
(12) Patent Application Publication
(10) Pub. No.: US 2010/0279413 A1
(43) Pub. Date: Nov. 4, 2010

(54) APPLICATIONS OF UBIQUINONES AND UBIQUINOLS

(75) Inventor: Randi Fain, Livingston, NJ (US)

Correspondence Address: MORGAN, LEWIS & BOCKIUS LLP (SF), One Market, Spear Street Tower, Suite 2800, San Francisco, CA, 94105, US

(73) Assignee: ZYMES, LLC

(21) Appl. No.: 12/598,400

(22) PCT Filed: Apr. 30, 2008
(86) PCT No.: PCT/US08/62123

§ 371 Date: Jan. 28, 2010

Related U.S. Application Data
(60) Provisional application No. 61/020,962, filed on Jan. 14, 2008.
(60) Provisional application No. 61/024,887, filed on Jan. 30, 2008.

(51) Int. Cl. C12N 5/0793; C12N 5/079; C12N 5/071

(52) U.S. Cl. 435/406; 435/404; 435/408; 435/325

(57) ABSTRACT

The present invention relates to cell culture medium compositions comprising lipophilic compounds and solubilizing agent and methods of using such compositions for the culturing of cells. The invention also relates to methods of treating disorders of the nervous system such as peripheral neuropathy.

### CROSS REFERENCES TO RELATED APPLICATIONS

This application claims priority under 35 U.S.C. §119(e) to U.S. Provisional Patent Application No. 60/915,061 filed on Apr. 30, 2007, U.S. Provisional Patent Application No. 61/020,962 filed Jan. 14, 2008 and U.S. Provisional Patent Application No. 61/024,887 filed Jan. 30, 2008, the disclosures of which is incorporated by reference herein in its entirety for all purposes.

### FIELD OF THE INVENTION

The present invention relates to cell culture medium compositions comprising lipophilic compounds and solubilizing agent and methods of using such compositions for the culturing of cells. The invention also relates to the prevention and/or treatment of diseases of the nervous system by administration to a subject in need thereof with a pharmaceutical formulation including an effective amount of a ubiquinone and/or a ubiquinol mixed with an agent that renders the ubiquinone and/or a ubiquinol soluble in water.

### BACKGROUND OF THE INVENTION

Many kinds of cells can be grown in culture, which have been used to study genetic, physiological, and other phenomena, as well as to manufacture certain macromolecules using various fermentation techniques known in the art. Cell culture media provide nutrients for maintaining and/or growing cells in a controlled, artificial and in vitro environment. Characteristics and compositions of the cell culture media vary depending on the particular cellular requirements and any functions for which the cells are cultured. Important parameters generally include osmolality, pH, and nutrient formulations. The normal environment of a cell in culture is an aqueous medium in which nutrients and other culture components are dissolved or suspended. Especially advantageous is incorporation of useable quantities of lipid soluble or other components that are only sparsely soluble in water.

Media formulations have been used to cultivate a number of cell types including animal, plant and bacterial cells. Cells cultivated in culture media catabolize available nutrients and produce useful biological substances such as monoclonal antibodies, hormones, growth factors, viruses, antigenic factors, enzymes, cytokines and the like. Such products have industrial and/or therapeutic applications. With the advent of recombinant DNA technology, cells can be engineered to produce large quantities of these products. Thus, the ability to cultivate cells in vitro is not only important for the study of cell physiology, but is also necessary for the production of useful substances which may not otherwise be obtained by cost-effective means.

It is estimated that there are more than 370 new biotechnology medicines in the pipeline. Producing biotech drugs is a complicated and time-consuming process. Cells must be grown in large stainless-steel fermentation vats under strictly maintained and regulated conditions. In some cases the proteins are secreted by the cells; in other cases the cells must be broken open so the protein can be extracted and purified. Once the method is tested, devised and scaled up, the biotech medicines can be produced in large batches. This is done by growing host cells that have been transformed to contain the gene or antibody of interest in carefully controlled conditions in large stainless-steel tanks The cells are kept alive and stimulated to produce the target proteins through precise culture conditions that include a balance of temperature (which can often vary by no more than one degree Celsius), oxygen, acidity (if pH levels change by even a small fraction, cells can easily die), media components and other variables. After careful culture in the appropriate media with or without serum (the duration varies depending on the protein produced and the nature of the organism), the proteins are isolated from the cultures, stringently tested at every step of purification, and formulated into pharmaceutically-active products.

Cell culture media supplemented with compounds having therapeutic efficacy provides a test environment suitable for determining the effect of these compounds on cell viability, growth, division, integrity, etc. Accordingly, conclusions can be drawn regarding the efficacy of therapeutic compounds on the treatment of a disease of a cell from the culture of that cell in the culture medium supplemented with the therapeutic compound. Thus, cell culture medium that includes a therapeutic compound solubilized in the aqueous medium is of use to investigate the efficacy of the therapeutic compound in treating, ameliorating or preventing a disease afflicting the cell or cell type in the culture.

Thus, the development of novel cell culture media, allowing for the ready and reliable inclusion of therapeutic compounds, particularly water-insoluble compounds, is key to continued development in the art of cell culture and to providing a platform for investigation of the therapeutic utility of both new and existing therapeutics on a wide rang of cell types.

### SUMMARY OF THE INVENTION

Surprisingly, the present invention provides a novel aqueous cell culture medium including compounds solubilized in the medium, which are generally considered to be essentially insoluble in aqueous media. Essentially any therapeutic agent can be incorporated into the media, though the media is considered to have particular utility with respect to agents that are essentially insoluble or only sparingly soluble in aqueous media. The invention is exemplified by reference to the essentially water-insoluble ubiquinones, particularly coenzyme Q10. The focus on this coenzyme is intended to exemplify, and not limit, the invention. Those of skill will appreciate that the cell culture media of the invention.

In an exemplary embodiment, the invention provides a water-soluble cell culture medium supplement composition comprising a) a solubilizing agent; and b) a ubiquinone/ubiquinol having a structure which is described herein. An exemplary ubiquinone/ubiquinol of use in the invention has a structure which is a member selected from Formula (I) and Formula (II):

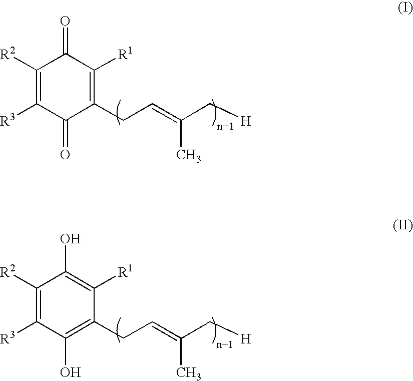

wherein the index n is an integer from 0 to 20. In exemplary embodiments, n ranges from 1 to 13. Exemplary ubiquinols/ubiquinones include CoQ₁₀, ubiquinol-50, or reduced CoQ₁₀. R¹, R² and R³ are members independently selected from H, substituted or unsubstituted alkyl and substituted or unsubstituted alkoxy. In various embodiments, R¹ is methyl. In exemplary embodiments, R² and R³ are methoxy.

In various embodiments, the solubilizing agent has a structure according to the following formula:

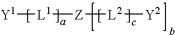

wherein a, b and c are integers independently selected from 0 and 1. Z is a hydrophobic moiety which is a member selected from tocopherol, sterol, ubiquinone and ubiquinol. Y¹ and Y² are hydrophilic moieties, which are members selected from polyethers, polyalcohols and derivatives thereof; and L¹ and L² are linkers.

In some embodiments, the solubilizing agent is polyoxyethanyl α-tocopheryl sebacate (PTS), polyoxyethanyl β-sitosteryl sebacate (PSS) or polyoxyethanyl-ubiquinol-sebacate (PQS).

The cell culture supplement can be prepared separately, or combined with other culture media, such a basal culture medium, for example, RPMI 1640, Dulbecco's modified

Eagle's Medium, and Ham's F12. Similarly, the pharmaceutical formulation including solubilized therapeutic (e.g., ubiquinone and/or ubiquinol) can be prepared and optionally combined with other components of the pharmaceutical formulation (dispersants, diluents, carriers, solvents, etc.).

The cell culture supplements and culture media provided herein are of use to culture a variety of cells, e.g., neuron, glia (e.g. Schwann cell) and Chinese Hamster Ovary (CHO) cells. The cell can be a primary cell culture cell, or derived from a cell line.

In addition to Applicants' unique discovery that ubiquinones and ubiquinols, particularly those solubilized in an aqueous medium, enhance the growth, longevity and robustness of nervous system cells, it has been discovered that solubilized ubiquinols are efficacious in the treatment, amelioration and prevention of peripheral neuropathy. Accordingly, the present invention also pertains to pharmaceutical compositions containing, in an aqueous medium, solubilized ubiquinols, e.g., coenzyme Q, brought into aqueous solution with one or more solubilizing agents. Also provided are methods of using such compositions for the prevention and treatment of peripheral neuropathy.

### BRIEF DESCRIPTION OF THE DRAWINGS

FIG. 1 shows that coenzyme Q10 (H_{Q}O™) promotes neurite growth. FIG. 1A depicts the concentration effect curve for Coenzyme Q10 to promote DRG neurite growth over 22 days in culture. FIG. 1B depicts the concentration effect curve for Coenzyme Q10 to promote DRG neurite growth over 11 days in culture. FIG. 1C depicts the concentration effect curve for Coenzyme Q10 to promote DRG neurite growth on day 11 in culture. FIG. 1D shows enhanced growth starting at day 8 out to day 22.

FIG. 2 shows that Coenzyme Q₁₀ (H_{Q}O™) reduces neurotoxicity induced by exposure to NRTIs.

### DETAILED DESCRIPTION OF THE INVENTIONS

### Definitions and Abbreviations

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization are those well known and commonly employed in the art. Standard techniques are used for nucleic acid and peptide synthesis. The techniques and procedures are generally performed according to conventional methods in the art and various general references, which are provided throughout this document. The nomenclature used herein and the laboratory procedures in analytical chemistry, and organic synthetic chemistry described below are those well known and commonly employed in the art. Standard techniques, or modifications thereof, are used for chemical syntheses and chemical analyses.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′—represents both —C(O)₂R′—and —R′C(O)₂—.

In general, an “acyl substituent” is also selected from the group set forth above. As used herein, the term “acyl subsituent” refers to groups attached to, and fulfilling the valence of a carbonyl carbon that is either directly or indirectly attached to the polycyclic nucleus of the compounds of the present invention.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroakyl substituents,” respectively, and they can be one or more of a variety of groups selected from, but not limited to: —OR′, ═O, =NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R′R″)═NR″, —NR—C(NR′R″)=NR′″, —S(O)R′, —S(O)₂R′, —S(O)₂NR′R″, —NRSO₂R′, —CN and —NO₂ in a number ranging from zero to (2 m′+1), where m′ is the total number of carbon atoms in such radical. R′, R″, R′″ and R′′″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R′′″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, the aryl substituents and heteroaryl substituents are generally referred to as “aryl substituents” and “heteroaryl substituents,” respectively and are varied and selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)=NR′″, —S(O)R′, —S(O)₂R′, —S(O)₂NR′R″, —NRSO₂R′, —CN and —NO₂, —R′, —N₃, —CH(Ph)₂-fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′ and R′ groups when more than one of these groups is present.

Two of the aryl substituents on adjacent atoms of the aryl or heteroaryl ring may optionally be replaced with a substituent of the formula -T—C(O)—(CRR)_{q}—U—wherein T and U are independently —NR—, —O—, —CRR′—or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂ or —S(O)₂NR′—. The substituents R, R′, R″ and R′″ are preferably independently selected from hydrogen or substituted or unsubstituted (C₁-C₆)alkyl.

As used herein, the term “heteroatom” includes oxygen (O), nitrogen (N), sulfur (S), phosphorus (P) and silicon (Si).

The term “labeling moiety” refers to a moiety, which provides a signal that is detectable by a detection method known in the art. The signal can be used to determine the location or concentration of the labeling moiety, for example, in an organism, a tissue sample or a reaction vial. Exemplary signals include color, emitted light of any wavelength, radioactivity, or any other electromagnetic or quantum mechanical effect. Exemplary labeling moieties include but are not limited to fluorescent molecules (e.g. fluorescein), luminescent moieties (e.g., transition-metal complexes), chemoluminescent molecules, molecules used in colorimetric applications (i.e. dye molecules), histochemical staining reagents, photoaffinity labels, magnetic resonance imaging (MRI) agents, radioactive labels, radiotracers and agents used in positron emission tomography (PET).

The term “targeting moiety” refers to a moiety which is capable of binding to a particular tissue- or cell-type (e.g., tumor cells, neuronal or glial cells, liver cells, Chinese Hamster Ovary (CHO) cells and the like) with at least some level of specificity. Exemplary targeting moieties are selected from carbohydrates, proteins, peptides, antibodies, and small-molecule ligands. In an exemplary embodiment, the targeting moiety is a ligand for a biological receptor, such as a cell surface receptor. In another exemplary embodiment, the targeting moiety is an antibody that is capable of binding to an antigen, such as a tissue- or tumor-specific antigen.

The term “drug moiety” refers to pharmaceutical drugs and other biologically active molecules. “Drug moiety” includes small-molecule drugs as well as biologics, including peptides, mutant and wild-type polypeptides, mutant and wild-type proteins, antibodies (e.g., humanized, monoclonal antibodies) and the like.

The term “water-soluble” refers to moieties that have a detectable degree of solubility in water. Methods to detect and/or quantify water solubility are well known in the art. Exemplary water-soluble polymers include peptides, saccharides, poly(ethers), poly(amines), poly(carboxylic acids) and the like. Peptides can have mixed sequences of be composed of a single amino acid, e.g., poly(lysine), poly(aspartic acid), and poly(glutamic acid). An exemplary polysaccharide is poly(sialic acid). An exemplary poly(ether) is poly(ethylene glycol), e.g., m-PEG. Poly(ethylene imine) is an exemplary polyamine, and poly(acrylic) acid is a representative poly(carboxylic acid).

The term “cell culture” or “culture” refers to the maintenance of cells in an artificial, e.g., an in vitro environment. It is to be understood, however, that the term “cell culture” is a generic term and may be used to encompass the cultivation not only of individual prokaryotic (e.g., bacterial) or eukaryotic (e.g., animal, plant and fungal) cells, but also of tissues, organs, organ systems or whole organisms, for which the terms “tissue culture,” “organ culture,” “organ system culture” or “organotypic culture” may occasionally be used interchangeably with the term “cell culture.”

The term “cultivation” refers to the maintenance of cells in an artificial environment under conditions favoring growth, differentiation, biologic production or continued viability, in an active or quiescent state, of the cells. Thus, “cultivation” may be used interchangeably with “cell culture” or any of its synonyms described above.

The term “cell culture medium,” “culture medium” (plural “media” in each case) and “medium formulation” refer to a nutritive solution that supports the cultivation and/or growth of cells; these phrases may be used interchangeably.

The term “contacting” refers to the placing of cells to be cultivated into a culture vessel with the medium in which the cells are to be cultivated. The term “contacting” encompasses inter alia mixing cells with medium, perfusing cells or organs with medium, pipetting medium onto cells in a culture vessel, and submerging cells in culture medium.

The term “culture vessel” refers to a glass, plastic, or metal container that can provide an aseptic environment for culturing cells.

The term “basal medium” refers to a solution of amino acids, vitamins, salts, and nutrients that is effective to support the growth of cells in culture, although normally these compounds will not support cell growth unless supplemented with additional compounds. The nutrients include a carbon source (e.g., a sugar such as glucose) that can be metabolized by the cells, as well as other compounds necessary for the cells' survival. These generally are compounds that the cells themselves could not synthesize, due to the absence of one or more of the gene(s) that encode the protein(s) necessary to synthesize the compound (e.g., essential amino acids) or, with respect to compounds which the cells can synthesize, because of their particular developmental state the gene(s) encoding the necessary biosynthetic proteins are not being expressed as sufficient levels. A number of base media are known in the art of mammalian cell culture, such as Dulbecco's Modified Eagle Media (DMEM), Knockout-DMEM (KO-DMEM), and DMEM/F12.

The term “conditioned medium” refers to a growth medium that is further supplemented with soluble factors derived from cells cultured in the medium. Techniques for isolating conditioned medium from a cell culture are well known in the art. As will be appreciated, conditioned medium is preferably essentially cell-free. In this context, “essentially cell-free” refers to a conditioned medium that contains fewer than about 10%, preferably fewer than about 5%, 1%, 0.1%, 0.01%, 0.001%, and 0.0001% than the number of cells per unit volume, as compared to the culture from which it was separated.

The term “defined medium” refers to a biochemically defined formulation comprised solely of the biochemically-defined constituents. A defined medium may include solely constituents having known chemical compositions. A defined medium may also include constituents that are derived from known sources. For example, a defined medium may also include factors and other compositions secreted from known tissues or cells; however, the defined medium will not include the conditioned medium from a culture of such cells. Thus, a “defined medium” may, if indicated, include particular compounds added to form the culture medium, up to and including a portion of a conditioned medium that has been fractionated to remove at least one component detectable in a sample of the conditioned medium that has not been fractionated. Here, to “substantially remove” of one or more detectable components of a conditioned medium refers to the removal of at least an amount of the detectable, known component(s) from the conditioned medium so as to result in a fractionated conditioned medium that differs from an unfractionated conditioned medium in its ability to support the long-term substantially undifferentiated culture of primate stem cells. Fractionation of a conditioned medium can be performed by any method (or combination of methods) suitable to remove the detectable component(s), for example, gel filtration chromatography, affinity chromatography, immune precipitation, etc. Similarly, or a “defined medium” may include serum components derived from an animal, including human serum components. In this context, “known” refers to the knowledge of one of ordinary skill in the art with reference to the chemical composition or constituent.

The term “complete medium” refers to a medium that provides all the necessary nutrients for a cell to grow in a cell culture.

It should be understood that there might be some over-lap between the different types of media described herein. For example, a serum-free medium may also be a defined medium, though some serum-free medium contain discrete proteins or bulk proteins.

The term “powder” refers to a composition that is present in granular form, which may or may not be complexed or agglomerated with a solvent such as water or serum. The term “dry powder” may be used interchangeably with the term “powder;” however, “dry powder” as used herein simply refers to the gross appearance of the granulated material and is not intended to mean that the material is completely free of complexed or agglomerated solvent unless otherwise indicated.

The term “ingredient” refers to any compound, whether of chemical or biological origin, that can be used in cell culture media to maintain or promote the growth of proliferation of cells. The terms “component,” “nutrient” and ingredient” can be used interchangeably and are all meant to refer to such compounds. Typical ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

The term “cytokine” refers to a compound that induces a physiological response in a cell, such as growth, differentiation, senescence, apoptosis, cytotoxicity, synthesis or transport, immune response or antibody secretion. Included in this definition of “cytokine” are growth factors, interleukins, colony-stimulating factors, interferons, thromboxanes, prostaglandins, hormones and lymphokines

The term “extract” refers to a composition comprising a purified, partially purified or concentrated preparation of the subgroups of a substance, typically formed by treatment of the substance either mechanically (e.g., by pressure treatment) or chemically (e.g., by distillation, solublization, precipitation, enzymatic action or high salt treatment).

The term “enzymatic digest” refers to a composition comprising a specialized type of extract, namely one prepared by treating the substance to be extracted (e.g., plant components or yeast cells) with at least one enzyme capable of breaking down the components of the substance into simpler forms (e.g., into a preparation comprising mono- or disaccharides and/or mono-, di- or tripeptides). In this context, and for the purposes of the present invention, the term “hydrolysate” may sometimes be used interchangeably with the term “enzymatic digest.”

The term “lipid” has its meaning as generally understood in biochemistry. “Lipid” also refers to a portion of the cell or an ingredient of a medium that is soluble in non-polar or non-aqueous solvent. The lipid may be sparsely soluble or insoluble in water in the presence or absence of other medium ingredients. Lipid may be soluble in a solvent mixture that includes water and one or more organic solvents. Lipids may comprise fatty acids, hormones, metabolites, cytokines, vitamins, indicators, stimulators or inhibitors. “Lipid” in some contexts may refer to ingredients that are normally insoluble or sparsely soluble in water, but that have been converted, e.g., by saponification hydroxylation, etc., to a form a compound or ion that is water soluble. Thus, for example, a fatty acid is a lipid, but also a salt of a fatty acid is to be included in the definition. Additionally, “lipid” is used generically to mean generally any component that is advantageously introduced using organic or non-polar solvents or that is not normally soluble in water or aqueous media. Lipids may be present as dissolved molecules, or in other forms such as micelles or other loose associations of molecules. A lipid may be used as a free molecule or may be bound to one or more other molecules. For example, proteins or peptides may be associated with one or more other lipids for stability and/or to aid in delivery to the agglomerated powder. Lipid may also refer to an ingredient that might act as a drug to inhibit or activate one or more functions of a cell or cell component.

The term “combining” refers to the mixing or admixing of ingredients in a cell culture medium formulation. Combining can occur in liquid or powder form or with one or more powders and one or more liquids.

The terms “treatment”, “treating” and the like generally refers to obtaining a desired pharmacologic, physiologic or cosmetic effect. The effect may be prophylactic in terms of completely or partially preventing a condition, appearance, disease or symptom thereof and/or may be therapeutic in terms of a partial or complete cure for a condition and/or adverse effect attributable to a condition or disease. “Treatment” as used herein covers any treatment of a condition, disease or undesirable appearance in a mammal, particularly a human, and includes one or more of the following: (a) preventing the disease (e.g. cancer), condition (pain) or appearance (e.g. wrinkles) from occurring in a subject which may be predisposed to such but has not yet been observed or diagnosed as having it; (b) inhibiting the disease, condition or appearance, i.e., causing regression of condition or appearance; and (c) relieving the disease, condition or undesired appearance, i.e., causing regression of condition or appearance.

“Peripheral neuropathy,” as used herein, refers to damage to nerves of the peripheral nervous system (PNS). Peripheral neuropathies may either be symmetrical and generalized or focal and multi-focal, which is usually a good indicator of the cause of the peripheral nerve disease.

Generalized peripheral neuropathies are symmetrical, and usually due to various systematic illnesses and disease processes that affect the peripheral nervous system in its entirety. They are further subdivided into three categories: Distal axonopathies, Myelinopathies, and Neuronopathies.

Distal axonopathies are the result of some metabolic or toxic derangement of neurons. They may be caused by metabolic diseases such as diabetes, renal failure, deficiency syndromes such as malnutrition and alcoholism, or the effects of toxins or drugs.

Myelinopathies are due to a primary attack on myelin causing an acute failure of impulse conduction. The most common cause is acute inflammatory demyelinating polyneuropathy (AIDP; also known as Guillain-Barrésyndrome). Other causes include chronic inflammatory demyelinating polyneuropathy (CIDP), genetic metabolic disorders (e.g., leukodystrophy), or toxins.

Neuronopathies are the result of destruction of PNS neurons. They may be caused by motor neuron diseases, sensory neuronopathies (e.g., Herpes zoster), toxins or autonomic dysfunction. Neurotoxins may cause neuronopathies, such as the chemotherapy agent vincristine.

Peripheral neuropathy can also be categorized by cause, such as being associated with systematic disease, being genetically acquired (such as inherited), or by chemical toxic agent, with the latter including antiretroviral toxic neuropathy (ATN). Peripheral neuropathy may be caused either by diseases of the nerve or from the side-effects of systemic illness, most often manifested as one or a combination of motor, sensory, sensorimotor, or autonomic neural dysfunction.

Chemotherapeutic agents known to cause sensory and/or motor neuropathies include vincristine, an antineoplastic drug used to treat haematological malignancies and sarcomas. The neurotoxicity is dose-related, and exhibits as reduced intestinal motility and peripheral neuropathy, especially in the distal muscles of the hands and feet, postural hypotension, and atony of the urinary bladder. Similar problems have been documented with taxol and cisplatin. Mollman, New Eng Jour Med. 322:126-127 (1990), although cisplatin-related neurotoxicity can be alleviated with nerve growth factor (NGF), Apfel et al., Annals of Neurology 31:76-80 (1992)). Although the neurotoxicity is sometimes reversible after removal of the neurotoxic agent, recovery can be a very slow process. Legha, Medical Toxicology 1:421-427 (1986); Olesen et al., Drug Safety 6:302-314 (1991).

There are also a number of inherited peripheral neuropathies, including Refsum's disease, Abetalipoproteinemia, Tangier disease, Krabbe's disease, Metachromatic leukodystrophy, Fabry's disease, Dejerine-Sottas syndrome, and others. The most common inherited neuropathy is Charcot-Marie-Tooth (CMT) Disease (also known as Peroneal Muscular Atrophy, or Hereditary Motor Sensory Neuropathy (HMSN)), which is also the most common hereditary neurological disorder. It is characterized by weakness and atrophy, primarily of the peroneal muscles, due to segmental demyelination of peripheral nerves and associated degeneration of axons and anterior horn cells. Autosomal dominant inheritance is usual, and associated degenerative CNS disorders, such as Friedreich's ataxia, are common. There are two primary forms of CMT Disease. Type I (70% of cases) was believed to have demyelination as its initial pathophysiology, but distal clinical involvement suggests a primary axonal degeneration, as in Type II. Type II (30% of cases) is primarily an axonal degeneration without demyelination, and may not be as severe as Type I. Nerve conduction impairment is often present at birth, though this is not a predictor of the possible age of onset or severity of progression. There are also very rare, Type III and Type IV forms, which are recessively-linked.

### Cell Culture Medium Supplement

In one aspect, the present invention provide a cell culture medium and medium supplements comprising a lipophilic compound (such as ubiquinone or ubiquinol) and a solubilizing agent. These supplements enhance the growth and survival of cultured cells when cells are cultured in their presence.

Methods and media formulations for cell culture have been developed for many different types of cells from different organisms. However, the conditions for cell culture generally are different from the conditions of the native microenvironments where such cells normally reside in a living organism. The difference, such as that of oxygen/carbon dioxide partial pressure and concentrations of nutrients, vitamins, and trace elements, asserts stress on the growth and survival of cells in a culture media, particularly oxidative stress due to reactive oxygen species (ROS).

ROS include oxygen ions, free radicals and peroxides (both inorganic and organic).

They are generally very small molecules and are highly reactive due to the presence of unpaired valence shell electrons. ROSs form as a natural byproduct of the normal metabolism of oxygen and have important roles in cell signaling. However, during times of environmental stress ROS levels can increase dramatically, which can result in significant damage to cell structures. This culminates in a situation known as oxidative stress. The effects of ROS on cell metabolism have been documented in a variety of species, such as the roles in programmed cell death (apoptosis). Cells are normally able to defend themselves against ROS damage through the use of enzymes such as superoxide dismutases and catalases. Small molecule antioxidants such as ascorbic acid (vitamin-C), uric acid, and glutathione also play important roles as cellular antioxidants. Similarly, polyphenol antioxidants assist in preventing ROS damage by scavenging free radicals.

However, oxidative stress raises particular concerns in cell culture because cells are generally under stress already in culture and without the normal defense systems available in a living organism. One ROS, hydrogen peroxide, which is lethal to cells, has been found to be produced in Dulbecco's Modified Eagle's Medium (DMEM) as photoproducts. (Wang et al., In Vitro 14(8):715-722 (1978)). Riboflavin and tryptophan or tyrosine are components necessary for formation of hydrogen peroxide during light exposure. Since most mammalian culture media contain riboflavin, tyrosine and tryptophan, toxic photoproducts are likely produced in most cell culture media. WO/1996/017626 discloses adding coenzyme Q₁₀ to a cultured human T-cell line (CCRF-CEM), undifferentiated neuronal cell line PC 12, and human neuroblastoma cell line IMR-32. Without being bound by theory, it is believed including ubiquinone or ubiquinol, such as coenzyme Q₁₀, provides protective effect against oxidative stress, thus promotes cell growth and survival in cell culture.

The cell culture medium supplement can be in solid form or in solution. In some embodiments, the supplement is in a solid form, and can be dissolved prior to be added to culture medium such as a basal medium or a complete medium as described herein. In some embodiments, the supplement is in solution form, and can be added to a medium such as basal medium or complete medium directly. The amount of supplement needs to be added to the medium can vary, such as depending on the culture conditions and the type of cells being cultured. Optimal amount can be determined by running a series of pilot cultures in relative small scales.

When the medium supplement according to the present invention is advantageously used by adding it to an ordinary medium, it is desirable to dissolve the medium supplement in a small volume of the medium and then add it to the whole medium.

The cell culture media supplements provided by the present methods may be used to support the growth of a cell, which may be a bacterial cell, a fungal cell (particularly a yeast cell), a plant cell or an animal cell (particularly an insect cell, a nematode cell or a mammalian cell, most preferably a human cell), any of which may be a somatic cell, a germ cell, a normal cell, a diseased cell, a transformed cell, a mutant cell, a stem cell, a precursor cell or an embryonic cell.

In another aspect, the present invention provide as cell culture medium supplement provided further comprises an additive. The additives could be any compounds that could be used in cell culture, include but is not limited to growth factors. One more additives can be included in the supplement composition.

### Cell Culture Medium

According to another aspect, the invention provides cell culture medium formulation comprising an ubiquinone or ubiquinol and a solubilizing agent described herein, and an un-supplemented cell culture medium.

The compositions of the invention contain from about 5% to about 50% by weight of ubiquinone/ubiquinol. In an exemplary embodiment, the composition contains from about 10% to about 30% (w/w) ubiquinone/ubiquinol, preferably from about 15% to about 25% (w/w).

By “un-supplemented cell culture medium,” or “un-supplemented medium” here in is meant a medium does not contain a lipophilic compound and a solubilizing agent provided herein. Un-supplemented media include, but not limited to, basal media, defined-media, serum-free media, conditioned media, and complete media.

There are many varied types of cell culture media that can be used to support cell viability, for example DMEM medium (H. J. Morton, In Vitro, 6, 89/1970), F12 medium (R. G. Ham, Proc. Natl. Acad. Sci. USA, 53, 288/1965) and RPMI 1640 medium (J. W. Goding, J. Immunol. Methods, 39, 285/1980; JAMA 199, 519/1957). Such media (often called “basal media”), however, are usually seriously deficient in the nutritional content required by most animal cells. Typically, serum must be added to the basal media to overcome these deficiencies. Generally, fetal bovine serum (FBS), horse serum or human serum is used in significant concentrations.

While the use of FBS is desirable, and often necessary, for proper cell growth, it has several disadvantages. It is relatively expensive, and its use greatly increases the cost of cell culture. In addition, it is difficult to obtain serum with consistent growth characteristics. Further, the biochemical complexity of FBS can complicate the downstream processing of the proteins of interest, therefore raising the production costs.

Serum-free medium is an excellent alternative to standard serum-containing media for the cultivation of cells. It has several advantages, which include better definition of the composition, reduced contamination and lower cost. A serum-free medium having cultivation ability comparable to that of the conventional serum-containing medium has long been sought.

One strategy to develop serum-free media has been to supplement the basal media with appropriate nutrients to avoid the addition of FBS, without sacrificing cell growth and/or protein production. Examples of such components include bovine serum albumin (BSA) or human serum albumin (HSA); certain growth factors derived from natural (animal) or recombinant sources, including epidermal growth factor (EGF) or fibroblast growth factor (FGF); lipids such as fatty acids, sterols and phospholipids; lipid derivatives and complexes such as phosphoethanolamine, ethanolamine and lipoproteins; protein and steroid hormones such as insulin, hydrocortisone and progesterone; nucleotide precursors; and certain trace elements. See Cell Culture Methods for Molecular and Cell Biology, Vol. 1, Barnes, D. W., et al., eds., New York: Alan R. Liss, Inc., (1984), herein incorporated by reference in its entirety.

It is known that cholesterol and cholesterol-containing fractions obtained from bovine serum are useful to promote the growth of various organisms. J. Bacteriol., Vol. 135, pp. 818-827 (1978) describes the use of a cholesterol-containing bovine serum fraction in the growth of Mycoplasma pneumoniae and Mycoplasma arthritidis. J. Gen. Microbiology, Vol. 116, pp. 539-543 (1980) describes the use of USP cholesterol in the growth of Treponema hyodysenteriae. In Vitro, Vol. 17, No. 5, pp. 519-530 (1981) discloses that mixtures of high density lipoproteins and transferrin can be used to grow certain mammalian cells in the absence of serum.

Any basal medium known in the art can be used in the presented invention. Basal media that may be used include those commercially available from Invitrogen, Lonza, Sigma Chemical Co., Life Technologies, Inc., or Bio Whittaker Co. Any basal medium may be used. The basal medium composition comprises carbon sources assimilatable by cells, digestible nitrogen sources and inorganic salts. More specifically, for example, inorganic salts, amino acids, glucose, and vitamins are included. If necessary, a trace substance for nutritional stimulation and an effective trace substance such as a precursor can be included in the basal medium composition. More specifically, for example, MEM medium (H. Eagle, Science, 130, 432 (1959)), DMEM medium (R. Dulbecco, Virology, 8, 396 (1959)), RPMI 1640 medium (G. E. Moore, J. A. M. A., 199, 519 (1967)), Ham's F12 medium (R. G. Ham, Proc. Natl. Acad. Sci. U.S.A., 53, 288 (1965)), MCDB104 medium (W. L. Mckeehan, In Vitro, 13, 399 (1977)), and MCDB153 medium (D. M. Peehe, In Vitro, 16, 526 (1980)) can be used. More basal medium compositions are described in GIBCO Cell Culture Systems Catalog, 59-117 (Invitrogen, 2005), herein incorporated by reference by it entirety.

In some embodiments, the cell culture media provided herein are produced by adding a lipophilic compound and a solubilizing agent described herein to a serum-free medium known in the art. Serum-free media are described in GIBCO Cell Culture Systems Catalog, 7-55 (Invitrogen, 2005), and U.S. Patent Application Publication No. 20060073591 (particularly, 19-39), herein all incorporated by reference. Other media which can be appropriately used in the present invention include serum-free medium ASF104 (Ajinomoto Co., Inc.), serum-free medium SF-02 (Sanko Junyaku Co., Ltd.), serum-free medium Hybridoma-SFM (Lifetech Oriental), serum-free medium BIO-MPM-1 (Biological Industries), serum-free medium EX-CELL.TM. 302-HDP (JRH Biosciences), serum-free medium Cosmedium 001 (Cosmo Bio), and serum-free medium SFM-101 (Nissui Pharmaceutical Co., Ltd.).

The media that can be used by the present invention include cell culture media that support the growth of animal cells, plant cells, bacterial cells or yeast cells.

Examples of animal cell culture media that may be used in the present invention include, but are not limited to Chu (N6) basal salt mixture, DKW/Juglans basal salt mixture, Gamborg's B-5 basal salt mixture, Gamborg's B-5 basal salt mixture with minimal organics, Hoagland's No. 2 basal salt mixture, McCown's woody plant basal salt mixture, Murashige and Skoog basal salt mixture (MS), Quoirin and Lepoivre basal salt mixture, Schenk and Hildebrandt basal salt mixture, White's basal salt mixture. Also can be included in the media are vitamins, antibiotics and growth factor mixes.

Examples of animal cell culture media that may be used in the present invention include, but are not limited to, DMEM, RPMI-1640, MCDB 131, MCDB 153, MDEM, IMDM, MEM, M199, McCoy's 5A, Williams' Media E, Leibovitz's L-15 Medium, Grace's Insect Medium, IPL41 Insect Medium, TC-100 Insect Medium, Schneider's Drosophila Medium, Wolf & Quimby's Amphibian Culture Medium, cell-specific serum-free media (SFM) such as those designed to support the culture of keratinocytes, endothelial cells, hepatocytes, melanocytes, etc., F10 Nutrient Mixture and F12 Nutrient Mixture. Other media are available commercially (e.g., from Invitrogen Corporation, Carlsbad Calif., and Sigma; St. Louis, Mo.). Formulations for these media as well as many other commonly used animal cell culture media, media supplements and media subgroups are well-known in the art and may be found, for example, in the GIBCO Catalogue and Reference Guide (Invitrogen Corporation, Carlsbad, Calif.) and in the Sigma Animal Cell Catalogue (Sigma; St. Louis, Mo.), all incorporated herein be reference in their entireties.

The cell culture media provided herein may also include media supplements including but not limited to, animal sera (such as bovine sera (e.g., fetal bovine, newborn calf and calf sera), human sera, equine sera, porcine sera, monkey sera, ape sera, rat sera, murine sera, rabbit sera, ovine sera and the like), defined replacements such as LipoMAXO, OptiMAb®, Knock-Out™ (each available from Invitrogen Corporation, Carlsbad, Calif.), hormones (including steroid hormones such as corticosteroids, estrogens, androgens (e.g., testosterone) and peptide hormones such as insulin, cytokines (including growth factors (e.g., EGF, aFGF, bFGF, HGF, IGF-1, IGF-2, NGF and the like), interleukins, colony-stimulating factors, interferons and the like), neurotransmitters, lipids (including phospholipids, sphingolipids, fatty acids, cholesterol and the like), attachment factors (including extracellular matrix components such as fibronectin, vitronectin, laminins, collagens, proteoglycans, glycosaminoglycans and the like), and extracts of animal tissues, organs or glands (such as bovine pituitary extract, bovine brain extract, chick embryo extract, bovine embryo extract, chicken tissue or meat extract, achilles tendon and extracts thereof) and the like). Other media supplements that may be used by the present invention include a variety of proteins (such as serum albumins, particularly bovine or human serum albumins; immunoglobulins and fragments or complexes thereof; aprotinin; hemoglobin; haemin or haematin; enzymes (such as trypsin, collagenases, pancreatinin or dispase); lipoproteins; fetuin; ferritin; etc.), which may be natural or recombinant; vitamins; amino acids and variants thereof (including, but not limited to, L-glutamine and cystine), enzyme co-factors; polysaccharides; salts or ions (including trace elements such as salts or ions of molybdenum, vanadium, cobalt, manganese, selenium, and the like); and other supplements and compositions that are useful in cultivating cells in vitro that will be familiar to one of ordinary skill. These sera and other media supplements are available commercially (for example, from Invitrogen Corporation, Carlsbad, Calif., and Sigma Cell Culture, St. Louis, Mo.); alternatively, sera and other media supplements described above may be isolated from their natural sources or produced recombinantly by art-known methods that will be routine to one of ordinary skill (see Freshney, R. I., Culture of Animal Cells, New York: Alan R. Liss, Inc., pp. 74-78 (1983), and references cited therein; see also Harlow, E., and Lane, D., Antibodies: A Laboratory Manual, Cold Spring Harbor, N.Y.: Cold Spring Harbor Laboratory, pp. 116-120 (1988)), all incorporated herein be reference in their entireties.

The media of the present invention may also contain buffers, include but are not limited to, phosphate-buffered saline (PBS) formulations, Tris-buffered saline (TBS) formulations, HEPES-buffered saline (HBS) formulations, Hanks' Balanced Salt Solutions (HBSS), Dulbecco's PBS (DPBS), Earle's Balanced Salt Solutions, Puck's Saline Solutions, Murashige and Skoog Plant Basal Salt Solutions, Keller's Marine Plant Basal Salt Solutions, Provasoli's Marine Plant Basal Salt Solutions, and Kao and Michayluk's Basal Salt Solutions. Formulations for these buffers, which are commercially available, as well as for many other commonly used buffers, are well-known in the art and may be found for example in the GIBCO Catalogue and Reference Guide (Invitrogen Corporation, Carlsbad, Calif.), in the DIFCO Manual (DIFCO; Norwood, Mass.), and in the Sigma Cell Culture Catalogues for animal and plant cell culture (Sigma; St. Louis, Mo.), all incorporated herein be reference in their entireties.

The cell culture media provided herein can be produced by different approaches. In some embodiments, the cell culture medium supplements provided herein, and optionally other desirable additives, can be added to an un-supplemented medium to produce a cell culture medium suitable for the maintenance and/or growth of the cell. In some embodiments, the lipophilic compound and solubilizing agent can be mixed first, and mixed with other ingredients of an un-supplemented medium known in the art.

In some embodiments, a lipophilic compound (such as an ubiquinone or ubiquinol) and a solubilizing agent are mixed and to be in solid form. Such solid form is then added to a solid form of un-supplemented medium before being dissolved in water to produce a cell culture medium solution.

In some embodiments, a lipophilic compound (such as an ubiquinone or ubiquinol) and a solubilizing agent are mixed and to be in solid form. Such solid form is then added to a solution form of un-supplemented medium to produce a cell culture medium solution.

In some embodiments, a lipophilic compound (such as an ubiquinone or ubiquinol) and a solubilizing agent are mixed and dissolved in a solvent, such as water, to produce a solution. This solution is then mixed with a basal medium, either in solid form or in solution form, to produce cell to produce a cell culture medium solution.

### Pharmaceutical Formulations

According to another aspect, the invention provides pharmaceutical formulations for treatment or prevention of nervous system disorders, e.g. peripheral neuropathy, comprising an ubiquinone or ubiquinol and a solubilizing agent described herein. In an exemplary embodiment, the solubilizing agent is according to Formula (II).

In an exemplary embodiment, the pharmaceutical formulations are of use in preventing or reducing neurotoxicity attributable to an administered medicament (e.g., anti-cancer, anti-HIV chemotherapeutic).

The compositions of the invention contain from about 5% to about 50% by weight of ubiquinone/ubiquinol. In an exemplary embodiment, the composition contains from about 10% to about 30% (w/w) ubiquinone/ubiquinol, preferably from about 15% to about 25% (w/w).

In an exemplary embodiment, the formulation of the invention is encompassed within a soft gelatin (soft gel) capsule. The method of making soft gel is disclosed in U.S. Patent Application No. 60/886,212, which is herein incorporated by reference. The soft gel capsules of the invention include ubiquinone/ubiquinol from about 1% to about 30% by weight. In one embodiment the soft gel capsule includes from about 3% to about 20% (w/w), and preferably from about 5% to about 20% of active component, such as CoQ₁₀. Typically, the formulations include from about 10% to about 30% (w/w) ubiquinone/ubiquinol, from about 15% to about 40% (w/w) solubilizing agent (e.g., PQS), from about 30% to about 60% (w/w) lipophilic carrier (e.g., fish oil) and from about 1% to about 10% (w/w) viscosity enhancer (e.g., beeswax). In an exemplary embodiment, the soft gel capsule of the invention includes CoQ₁₀, PQS, beeswax and a lipophilic carrier (e.g., fish oil) enriched with omega fatty acid.

In an exemplary embodiment, the ubiquinone or ubiquinol is combined with solubilizing agents to obtain improved bioavailability. Such formulations may further contain additional active ingredients and/or pharmaceutically or cosmetically acceptable additives or vehicles, including solvents, adjuvants, excipients, sweeteners, fillers, colorants, flavoring agents, lubricants, binders, moisturizing agents, preservatives and mixtures thereof. The formulations may be suitable for topical (e.g., a cream, lotion, gel, ointment, dermal adhesive patch), oral (e.g., a capsule, tablet, caplet, granulate), or parenteral (e.g., suppository, sterile solution) administration. Among the acceptable vehicles and solvents that may be employed for administration by injection are water, mildly acidified water, Ringer's solution and isotonic sodium chloride solution.

Ubiquinone or ubiquinol, when combined with a solubilizing agent of the invention, may be administered to a warm-blooded animal, particularly a human, in need of the prophylaxis or therapy. The method comprises administering to such human or warm-blooded animal, an effective amount of a water-soluble composition including the solubilizing agent and ubiquinone or ubiquinol.

When the ubiquinol is linked to the hydrophilic moiety through a linker, which is cleavable in vivo, this composition provides an additional benefit for the patient. In vivo, the solubilizing agent is hydrolyzed by enzymes and is systemically converted back to the respective ubiquinol, which is further converted to the respective ubiquinone.

The dose of a ubiquinone or ubiquinol for treating peripheral neuropathy varies upon the manner of administration, the age, sex, the body weight of the subject, and the condition being treated, and will ultimately be decided by the attending physician or veterinarian. Such an amount of the ubiquinone or ubiquinol as determined by the attending physician or veterinarian is referred to herein as a “therapeutically effective amount”.

The compositions described herein are preferably pharmaceutical compositions formulated according to known methods. Formulations are described in detail in a number of sources, which are well known and readily available to those skilled in the art. For example, Remington's Pharmaceutical Science by E. W. Martin describes formulation, which can be used in connection with the subject invention. In general, the compositions of the subject invention are formulated such that an effective amount of coenzyme Q is provided in the composition.

In accordance with the present invention, pharmaceutical compositions are provided which comprise, an active ingredient as described, supra, and an effective amount of one or more pharmaceutically acceptable excipients, vehicles, carriers or diluents. Examples of such carriers (beside the ubiquinone or ubiquinol) include ethanol, dimethyl sulfoxide, glycerol, silica, alumina, starch, and equivalent carriers and diluents. Further, acceptable carriers can be either solid or liquid. Solid form preparations include powders, tablets, pills, capsules, cachets, suppositories and dispersible granules. A solid carrier can be one or more substances, which may act as diluents, flavoring agents, solubilizers, lubricants, suspending agents, binders, preservatives, tablet disintegrating agents or encapsulating materials.

Injectable preparations include sterile suspensions, solutions or emulsions of the active ingredient in aqueous or oily vehicles. The compositions can also contain formulating agents, such as suspending, stabilizing and/or dispersing agent. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, dextrose solution, etc., before use. To this end, the compositions can be lyophilized. The stored preparations can be supplied in unit dosage forms and reconstituted prior to use in vivo.

For prolonged delivery, the active ingredient can be formulated as a depot preparation, for administration by implantation; e.g., subcutaneous, intradermal, or intramuscular injection.

Alternatively, transdermal delivery systems manufactured as an adhesive disc or patch which slowly releases the active ingredient for percutaneous absorption can be used. To this end, permeation enhancers can be used to facilitate transdermal penetration of the active ingredient. A particular benefit can be achieved by incorporating the active agents of the invention into a nitroglycerin patch for use in patients with ischemic heart disease and hypercholesterolemia.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound.

For buccal administration, the compositions can take the form of tablets or lozenges formulated in conventional manner. For rectal and vaginal routes of administration, the active ingredient may be formulated as solutions (for retention enemas) suppositories or ointments.

For administration by inhalation, the active ingredient can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The disclosed pharmaceutical compositions can be subdivided into unit doses containing appropriate quantities of the active component. The unit dosage form can be a packaged preparation, such as packeted tablets, capsules, and powders in paper or plastic containers or in vials or ampoules. Also, the unit dosage can be a liquid based preparation or formulated to be incorporated into solid food products, chewing gum, or lozenges.

Pharmaceutically acceptable salts (counter ions) can be conveniently prepared by ion-exchange chromatography or other methods as are well known in the art.

### Ubiquinones/Ubiquinols

In one aspect, the invention provides for a cell culture composition comprising a solubilized (e.g., water solubilized) ubiquinone and/or ubiquinol having a structure which is a member selected from Formula (I) and Formula (II), and combinations thereof:

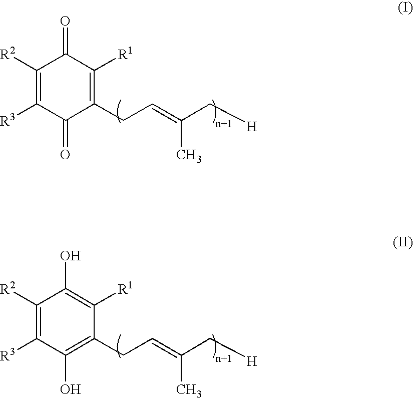

wherein the integer n is a member selected from 0 to 13. R₁, R² and R³ are members independently selected from H, substituted or unsubstituted alkyl and substituted or unsubstituted alkoxy. In various embodiments, n is 9. In exemplary embodiments, R¹ is methyl. Exemplary moieties for R² and R³ are methoxy. The ubiquinone can be CoQ₁₀ or ubiquinol-50, or reduced CoQ₁₀.

In an exemplary embodiment, the ubiquinone or ubiquinol is coenzyme Q (or its reduced form). Coenzyme Q refers to a series of quinones, which are widely distributed in animals, plants and microorganisms, and are found in various biological species differ only slightly in chemical structure and form a group of related, 2-3-dimethoxy-5-methyl-benzoquinones with a polyisoprenoid side chain in the 6-position, which varies in length from 30 to 50 carbon atoms. Since each isoprenoid unit in the chain contains five carbon atoms, the number of isoprenoid units in the side chain varies from 6 to 10. The different numbers of the groups have been designated by a subscript following the Q to denote the number of isoprenoid units in the side chain, as in Q₁₀ (e.g. coenzyme Q₁₀). Difference in properties is due to the difference in length of the side chain. The members of the group known to occur naturally are Q6 through Q₁₀.

Coenzyme Q functions as an agent for carrying out oxidation and reduction within cells. Its primary site of function is in the terminal electron transport system where it acts as an electron or hydrogen carrier between the flavoproteins (which catalyze the oxidation of succinate and reduced pyridine nucleotides) and the cytochromes. This process is carried out in the mitochondria of cells of higher organisms. As an electron carrier/acceptor, coenzyme Q₁₀ is continually going through an oxidation reduction cycle. As each coenzyme Q₁₀ molecule accepts electrons, it is reduced; when it gives up electrons, it becomes oxidized again. In coenzyme Q₁₀'s reduced form (ubiquinol), the coenzyme Q₁₀ molecule holds electrons loosely and will quite easily give up one or two electrons to neutralize free radicals.

In its electron rich reduced form, coenzyme Q₁₀ is as potent an antioxidant as vitamin E. Coenzyme Q₁₀'s main role as an antioxidant is in the mitochondria where it first participates in the process by which free radicals are generated and then helps to quench the extra free radicals that threaten cellular components such as DNA, RNA, proteins, and cell membranes. One of coenzyme Q₁₀'s key antioxidant actions is within the cell membrane, where it counters the oxidative attack of polyunsaturated lipids (lipid peroxidation), which causes damage in a self-propagating, destructive chain reaction that ultimately results in membrane degeneration leading to cell death.

The structure of coenzyme Q is provided below in both the oxidized and reduced forms. As will be apparent, the compositions of the invention can comprised a mixture or combination of oxidized and reduced coenzyme Q. As described above, exemplary ubiquinones and ubiquinols include a range of isoprenoid units, e.g., n is 6-10, for example, coenzyme Q₁₀.

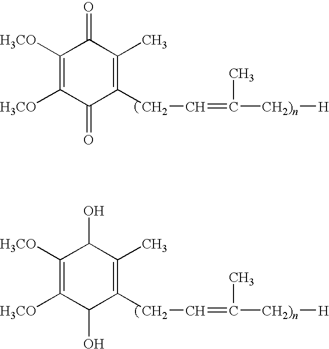

The method of making ubiquinones or ubiquinols is known in the art. There are two general processes by which coenzyme Q₁₀ (CoQ₁₀) can be produced, ‘fermentation’ or ‘synthesis’. Ubiquinones/ubiquinols can be purchased commercially from sources such as Kaneka (Japan) and Nisshin (Japan). Most of the world's supply of coenzyme Q₁₀ is generated in Japan using patented processes based on the fermentation of glucose by yeast and is then purified. See U.S. Pat. Nos. 4,447,362 and 4,194,065, all are incorporated herein by reference in their entirety.

The remaining supply of coenzyme Q₁₀ is made by what is referred to as the “synthetic” method. This method involves combining two naturally derived compounds. One is ‘solanesol’, which is isolated and purified from tobacco plants. The other component is TMT, derived from gallic acid, found in several different plant sources. Thus, the synthetic process of coenzyme Q₁₀ involves combining two naturally occurring compounds derived from plants. For example, organic chemical synthesis of coenzyme Q is described in U.S. Pat. No. 6,686,485. In particular is the Lipshutz method for the synthesis of ubiquinones and ubiquinone analogues that is described in U.S. Pat. Nos. 6,545,184, 6,852,895, and

U.S. patent application Ser. Nos. 10/992,270, 11/003,544, 11/304,023 and 10/581,566 and U.S. Provisional Patent Application No. 60/804,920, all are incorporated herein by reference in their entirety.

### Solubilizinz Agent

The formulations of lipophilic compounds of use in the invention include a solubilizing agent. In an exemplary embodiment, the solubilizing agent has a structure according to the following formula:

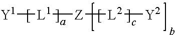

In Formula (III), a, b and c are integers independently selected from 0 and 1. In one example, b is 0. Z is a hydrophobic (lipophilic) moiety. In one example, Z is a sterol (e.g., beta-sitosterol, cholesterol). In another example, Z is a tocopherol (e.g., alpha-tocopherol, alpha-tocotrienol) or a derivative or homologue thereof. In yet another example, Z is an ubiquinol. A person of ordinary skill in the art will understand that the residue of the hydrophobic moiety is the entire hydrophobic molecule, except for at least one hydrogen atom, which is replaced with the hydrophilic moiety or the linker-hydrophilic moiety cassette (e.g., hydrogen atom of esterified hydroxyl group, such as 3-β-hydroxyl group of cholesterol or sitosterol or 6-hydroxyl group of α-tocopherol).

In Formula (III), Y¹ and Y² are linear or branched hydrophilic moieties comprising at least one polymeric moiety, wherein each polymeric moiety is independently selected. In one example, Y¹ and Y² are independently selected from hydrophilic (i.e., water-soluble) polymers. In another example, Y¹ and Y² are members independently selected from poly(alkylene oxides) (i.e., polyethers), polyalcohols, polysaccharides (e.g., polysialic acid), polyamino acids (e.g., polyglutamic acid, polylysine), polyphosphoric acids, polyamines and derivatives thereof. Exemplary poly(alkylene oxides) include polyethylene glycol (PEG) and polypropylene glycol (PPG). PEG derivatives include those, in which the terminal hydroxyl group is replaced with another moiety, such as an alkyl group (e.g., methyl, ethyl or propyl). In one example, the hydrophilic moiety is methyl-PEG (mPEG).

PEG is usually a mixture of oligomers characterized by an average molecular weight. In one example, the PEG has an average molecular weight from about 200 to about 5000. In another exemplary embodiment, PEG has an average molecular weight from about 500 to about 1500. In another exemplary embodiment, PEG has an average molecular weight from about 500 to about 700 or about 900 to about 1200. In one example, the lipophilic moiety of the solubilizing agent is PEG-400. In one example, the lipophilic moiety of the solubilizing agent is PEG-600. Both linear and branched PEG moieties can be used as the hydrophilic moiety of the solubilizing agent in the practice of the invention. In an exemplary embodiment, PEG has between 1000 and 5000 subunits. In an exemplary embodiment, PEG has between 100 and 500 subunits. In an exemplary embodiment, PEG has between 10 and 50 subunits. In an exemplary embodiment, PEG has between 1 and 25 subunits. In an exemplary embodiment, PEG has between 15 and 25 subunits. PEG has between 5 and 100 subunits. In an exemplary embodiment, PEG has between 1 and 500 subunits.

In a further embodiment the poly(ethylene glycol) is a branched PEG having more than one PEG moiety attached. Examples of branched PEGs are described in U.S. Pat. No. 5,932,462; U.S. Pat. No. 5,342,940; U.S. Pat. No. 5,643,575; U.S. Pat. No. 5,919,455; U.S. Pat. No. 6,113,906; U.S. Pat. No. 5,183,660 and WO 02/09766; as well as Kodera Y., Bioconjugate Chemistry 5: 283-288 (1994); and Yamasaki et al., Agric. Biol. Chem., 52: 2125-2127, 1998, all of which are incorporated herein by reference in their entirety. Exemplary branched PEG moieties involve a branched core molecule having at least two PEG arms attached, each at a different attachment point.

In an exemplary embodiment, at least one of Y¹ and Y² includes a moiety having the following structure:

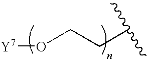

wherein Y⁷ is a member selected from CH₃ and H, and n is a member selected from 1 to 5000 (e.g., 1 to 2500). In an exemplary embodiment, n is a member selected from 1000-5000. In an exemplary embodiment, n is a member selected from 1-500. In an exemplary embodiment, n is a member selected from 5-100. In an exemplary embodiment, n is a member selected from 100-500. In an exemplary embodiment, n is a member selected from 10-50. In an exemplary embodiment, n is a member selected from 1-25.

In an exemplary embodiment, Y¹ and/or Y² is:

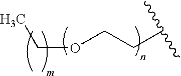

wherein m is a member selected from 0 to 30, and n is a member selected from 1 to 5000. In an exemplary embodiment, m is a member selected from 5-20. In an exemplary embodiment, m is a member selected from 8-15. In an exemplary embodiment, n is a member selected from 1000-5000. In an exemplary embodiment, n is a member selected from 100-500. In an exemplary embodiment, n is a member selected from 10-50. In an exemplary embodiment, n is a member selected from 1-25. In an exemplary embodiment, n is a member selected from 5-100. In an exemplary embodiment, n is a member selected from 1-500.

In an exemplary embodiment, Y¹ and/or Y² is:

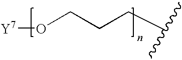

wherein Y⁷ is a member selected from CH₃ and H, and n is a member selected from 1 to 2500. In an exemplary embodiment, n is a member selected from 1000-5000. In an exemplary embodiment, n is a member selected from 100-500. In an exemplary embodiment, n is a member selected from 10-50. In an exemplary embodiment, n is a member selected from 1-25. In an exemplary embodiment, n is a member selected from 5-100. In an exemplary embodiment, n is a member selected from 1-500.

In an exemplary embodiment, Y¹ and/or Y² is:

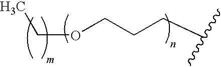

wherein m is a member selected from 0 to 30, and n is a member selected from 1 to 2500. In an exemplary embodiment, m is a member selected from 5-20. In an exemplary embodiment, m is a member selected from 8-15. In an exemplary embodiment, n is a member selected from 1000-5000. In an exemplary embodiment, n is a member selected from 100-500. In an exemplary embodiment, n is a member selected from 10-50. In an exemplary embodiment, n is a member selected from 1-25. In an exemplary embodiment, n is a member selected from 5-100. In an exemplary embodiment, n is a member selected from 1-500.

In one example, the hydrophilic molecule has a reactive functional group, which can be used to chemically attach the hydrophilic molecule to the hydrophobic moiety (e.g., sterol, tocopherol or ubiquinol), either directly or through a linker moiety. Examples of functional groups include esterifiable hydroxyl groups, carboxy groups and amino groups. In one example, the hydrophilic moiety is a polyether (e.g., polyalkylene glycol). The term “polyalkylene glycol” includes polymers of lower alkylene oxides, in particular polymers of ethylene oxide (polyethylene glycols) and propylene oxide (polypropylene glycols), having an esterifiable hydroxyl group at least at one end of the polymer molecule, as well as derivatives of such polymers having esterifiable carboxylic acid groups. The residue of the hydrophilic moiety is the entire hydrophilic molecule, except for the atom involved in forming the bond to the hydrophobic moiety or the linker moiety (i.e. hydrogen atom of an esterified hydroxyl group).

In Formula (III), L¹ and L² are linker moieties. In one example, L¹ and L² are independently selected from a single bond, substituted or unsubstituted alkyl, substituted or unsubstituted heteroalkyl, substituted or unsubstituted aryl, substituted or unsubstituted heteroaryl and substituted or unsubstituted heterocycloalkyl.

In one example, at least one of L¹ and L² includes a linear or branched C₂, C₃, C₄,

C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄ or C₂₅-C₃₀ alkyl chain, optionally incorporating at least one functional group. Exemplary functional groups according to this embodiment include ether, thioether, ester, carbonamide, sulfonamide, carbonate and urea groups.

In another example, at least one of L¹ and L² includes a moiety having the following formula:

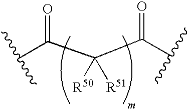

wherein m is an integer selected from 1 to 30. In one example, m is selected from 2 to 20. Each R⁵⁰ and each R⁵¹ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl.

In another example, at least one of L¹ and L² includes a moiety having the following formula:

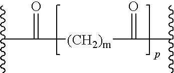

wherein m is an integer selected from 1 to 18 (e.g., from 1 to 10); and p is an integer selected from 0 and 1.

When p is 1, the linker can be derived from an alkanedioic acid of the general formula HOOC—(CH₂)ₘ—COOH. Preferred linkers include diesters derived from an alkanedioic acid. Forr the practice of the present invention, alkanedioic acids with m from 0 to 18 are preferred, those with m from 6 to 10 being particularly preferred. In some embodiments, sebacic acid (m=8) is particularly preferred. In another exemplary embodiment, the solubilizing agent includes the moiety:

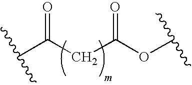

wherein m is a member selected from 4, 6, 8, 10, 12 and 14. In one example, m is 8 and the linker is derived from sebacic acid.

Other preferred linkers include diethers derived from a substituted alkane. In an exemplary embodiment the substituted alkane has the general structure X—(CH₂)ₙ—X′ wherein X and X′ independently represent a leaving group such as a halogen atom or a tosylate group. For the practice of the present invention, substituted alkanes with n from 0 to 18 are preferred, those with n from 6 to 10 being particularly preferred. The ether derived from a 1,10-substituted decane (n=10), such as 1,10-dibromodecane is most particularly preferred.

In yet another example, the solubilizing agent includes a moiety, which is a member selected from:

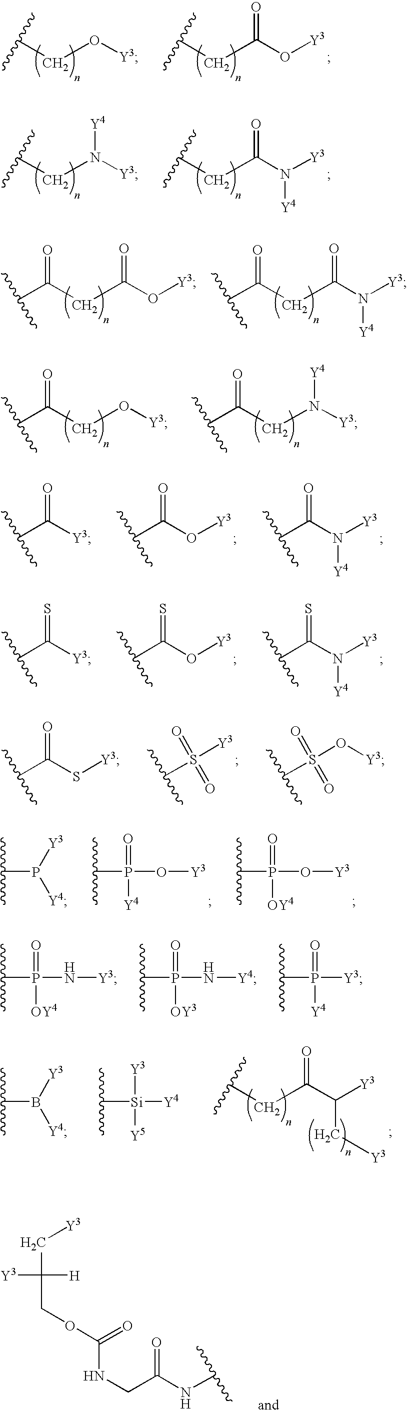

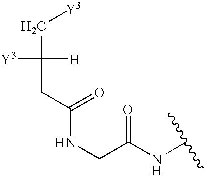

wherein the integer n is a member selected from 0 to 18. Y³ is a member selected from Y¹ and Y². Y⁴ and Y⁵ are members independently selected from H, substituted or unsubstituted alkyl, substituted or unsubstituted heteroalkyl, substituted or unsubstituted aryl, substituted or unsubstituted heteroaryl and substituted or unsubstituted cycloalkyl and substituted or unsubstituted heterocycloalkyl.

In an exemplary embodiment, the solubilizing agent includes a branched linker. In one example, at least one of L¹ and L² includes a moiety having the following formula:

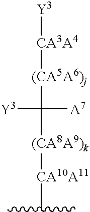

wherein in the integers j and k are independently selected from 0 to 20. A¹, A², A³, A⁴, A⁵, A⁶, A⁷, A⁸, A⁹, A¹⁰ and A¹¹ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —NA¹² A¹³, —OA¹² and —SiA¹² A¹³. A¹² and A¹³ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

In one embodiment the solubilizing agent is not PCS (polyoxyethanyl-cholesteryl sebacate). In another embodiment, the solubilizing agent is not TPGS (polyoxyethanyl-a-tocopheryl succinate).

In one exemplary embodiment, the solubilizing agent has a structure according to one of the following formulae:

Y¹—Z—Y²;

Y¹—L¹—Z—Y²;

Y¹—Z—L²—Y²; and

Y¹—L¹—Z—L²—Y²

wherein a, Y¹, Z and L¹ are defined as herein above. All embodiments described herein above for Formula (III) equally apply to the examples of this paragraph.

In one example, the solubilizing agent has a structure according to Formula (IV), wherein the integer a, Y¹, Z and L¹ are defined as herein above:

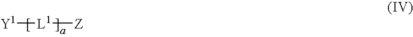

All embodiments described herein above for Formula (III) equally apply to the examples of this paragraph.

### Solubilizing Agents Wherein Z is a Sterol

In an exemplary embodiment, Z is a sterol. Exemplary sterols include 7-dehydrocholesterol, campesterol, sitosterol, ergosterol, stigmasterol, zoosterol and phytosterol. Cholesterol and sitosterol are preferred sterols, sitosterol being particularly preferred. In an exemplary embodiment, Z is member selected from a zoosterol and a phytosterol. In various embodiments, Z is a sterol with an oxygen atom at the 3-position of the A-ring. In an exemplary embodiment, Z is:

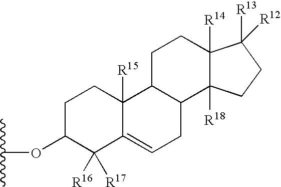

wherein at least one of R¹² and R¹³ is substituted or unsubstituted alkyl. R¹⁴, R₁₅, R₁₆, R₁₇, and R¹⁸ are independently H, or substituted or unsubstituted alkyl. In an exemplary embodiment, Z is a member selected from

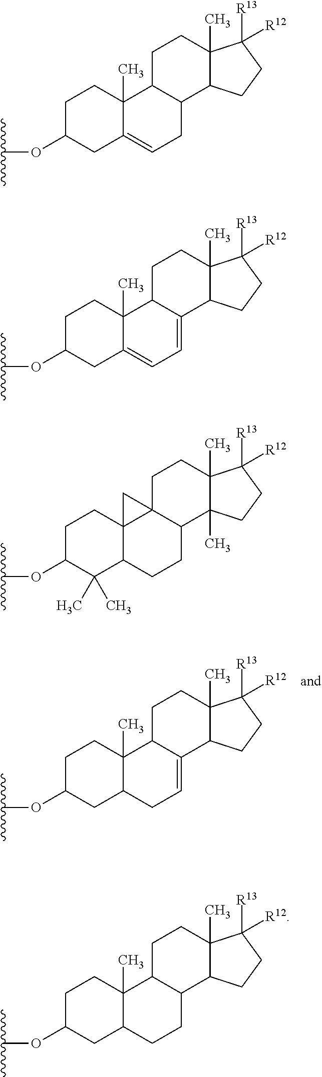

In an exemplary embodiment, Z is a member selected from:

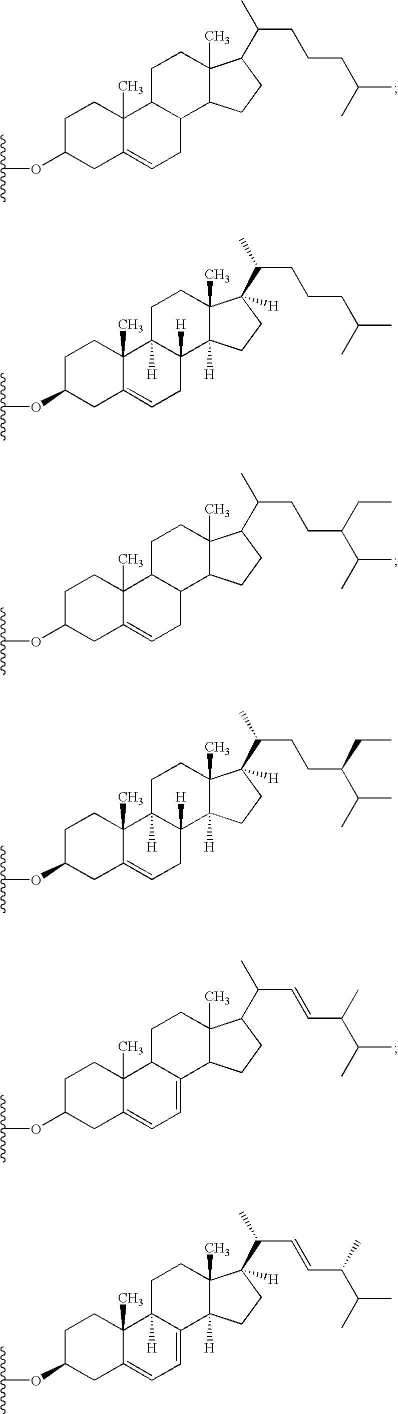

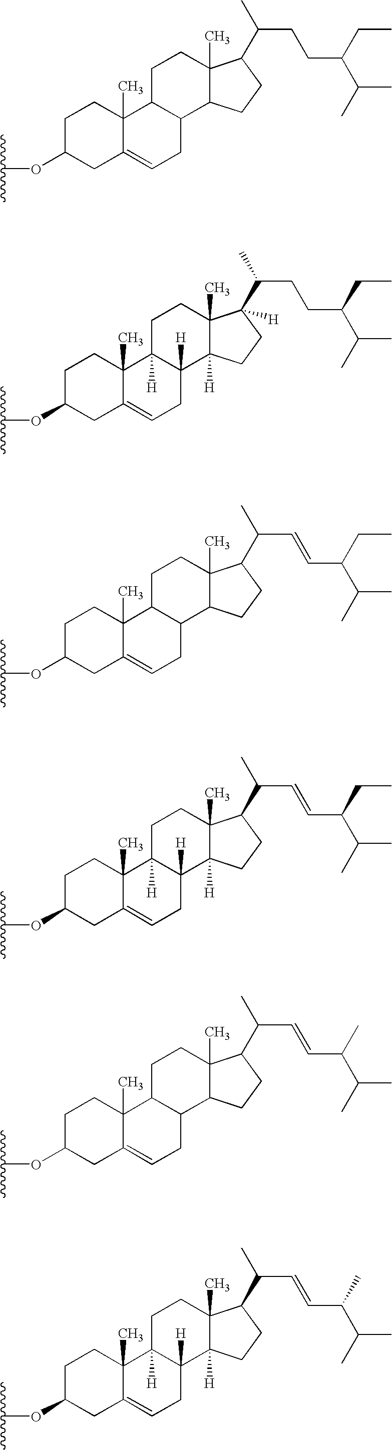

Additional, non-limiting examples of sterols include episterol, cycloartenol, avenasterol, 24-methylenecycloartenol.

### Solubilizing Agents Wherein Z is a Tocopherol or a Tocotrienol

In another embodiment, Z is a member selected from a substituted or unsubstituted tocopherol and a substituted or unsubstituted tocotrienol. In one example, Z is an α-β-, γ-, or Δ-tocopherol. Exemplary tocopherols of use include α-(+)-Tocopherol and α-(+)-tocopherol and DL tocopherol. In an exemplary embodiment, Z has a structure according to the following formula:

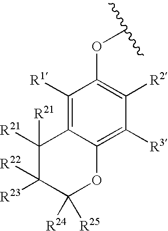

wherein R¹′, R²′ and R³′ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R²′ and R³′, together with the carbon atoms to which they are attached, are optionally joined to form a 5- to 7-membered ring. R²⁰, R²¹, R²², R²³, R²⁴ and R²⁵ are members selected from H, halogen, nitro, cyano, OR¹⁷, SR¹⁷, NR¹⁷R¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. In an exemplary embodiment, at least one of R²⁴ and R²⁵ comprises an isoprene moiety.

In another exemplary embodiment, R¹′, R²′ and R³′ are members independently selected from H and methyl. In another exemplary embodiment, R³′ is methyl, R²′ is methyl and R¹′ is methyl. In another exemplary embodiment, R³′ is methyl, R²′ is H and R¹′ is methyl. In another exemplary embodiment, R³′ is methyl, R²′ is methyl and R¹′ is H. In another exemplary embodiment, R³′ is methyl, R²′ is H and R¹′ is H.

In one example, Z has a structure according to the following formulae:

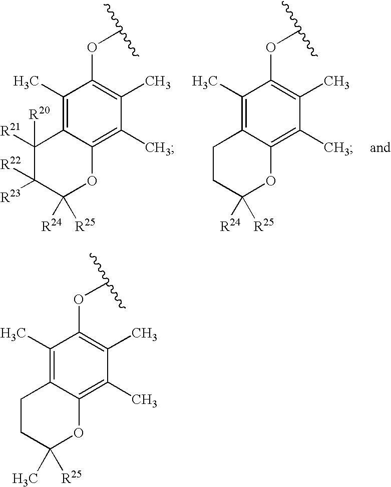

wherein R²⁵ is a member selected from substituted or unsubstituted alkyl and substituted or unsubstituted heteroalkyl. In one example, R²⁴ is methyl. In another example, R²⁵ includes a moiety having a structure selected from the following formulae:

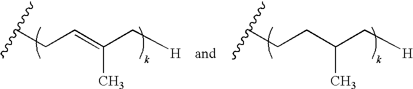

wherein k is an integer selected from 1 to 20. In an exemplary embodiment, k is from 2 to 12. In an exemplary embodiment, k is from 2 to 6. In another exemplary embodiment, k is 3.

In an exemplary embodiment, the solubilizing agent has a structure according to the following formula:

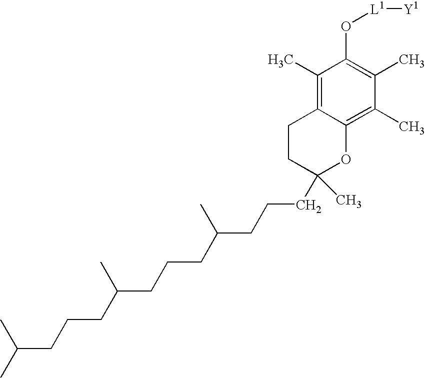

In an exemplary embodiment, the moiety L¹-Y¹ has a structure according to the following formula:

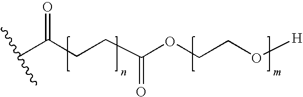

wherein n is member selected from 1 to 20, m is a member selected from 1 to 5000. In another exemplary embodiment, n is 4. In another exemplary embodiment, m is a member selected from 1 to 2,500.

### Solubilizing Agents Wherein Z is Ubiquinol

In an exemplary embodiment, Z is an ubiquinol. In an exemplary embodiment one or both of the phenolic hydroxy groups of the ubiquinol are derivatized with a hydrophilic moiety of the invention. In an exemplary embodiment, the solubilizing agent has a structure according to the Formula (V):

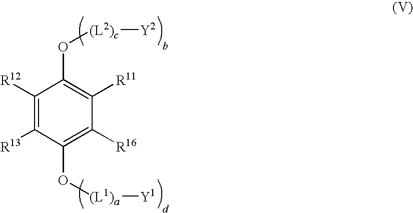

In Formula (V), L¹, L², Y¹ and Y² are defined as herein above. R₁₁, R₁₂, R¹³ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R¹⁶ is a member selected from OR¹⁷, SR¹⁷, NR¹⁷R¹⁸ substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R¹⁷ and R¹⁸ are members independently selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R¹² and R¹³, along with the atoms to which they are attached, are optionally joined to form a 4- to 8-membered ring.

In one example, L¹ and L² are linker moieties, which are members independently selected from substituted or unsubstituted alkyl and substituted or unsubstituted heteroalkyl. In another example, Y¹ and Y² are polymeric hydrophilic moieties, which are members independently selected from polyethers, polyalcohols and derivatives thereof. In one embodiment, Y¹, Y², L¹ and L² do not comprise a labeling moiety, a targeting moiety or a drug moiety. The indices a, b, c and d are members independently selected from 0 and 1 with the proviso that at least one of b and d is 1. When b is 0, ((L²)_{c}-Y²)_{b} is preferably a member selected from H, a negative charge, and a salt counterion. When d is 0, ((L¹)ₐ-Y¹)_{d} is preferably a member selected from H, a negative charge, and a salt counterion.

In an exemplary embodiment, in Formula (V), R¹⁶ includes a moiety having a structure selected from the following formulae:

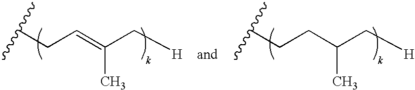

wherein k is an integer selected from 1 to 20. In an exemplary embodiment, k is an integer selected from 6, 7, 8, 9, 10, 11 and 12. In another exemplary embodiment, k is 10.

In an exemplary embodiment, in Formula (V), R¹¹, R¹² and R¹³ are members independently selected from H, unsubstituted alkyl (e.g., methyl, ethyl), alkoxy (e.g., methoxy, t-butoxy), halogen substituted alkoxy and halogen-substituted alkyl (e.g., CF₃). In one example, R¹¹ is H. In another embodiment of the invention, in Formula (V), R¹¹ is a methyl group.

In another exemplary embodiment, one or more of the substituents R¹¹, R¹² and R¹³ include halogen atoms. In another exemplary embodiment the halogen is fluoro. Exemplary fluoroalkyl and fluoroalkoxy groups according to this aspect of the invention include but are not limited to CF₃, OCF₃, CHF₂, OCHF₂, CH₂F, and OCH₂F.

In a particular example, R^{H} is methyl and R¹² and R¹³ are both methoxy. Hence, in an exemplary embodiment, the solubilizing agent has a structure according to the Formula (VI):

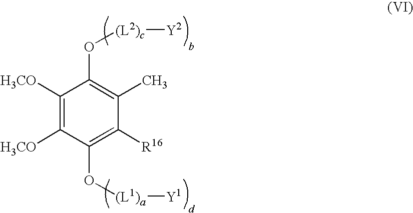

An exemplary solubilizing agent according to Formula (VI) has a structure according to Formula (VII):

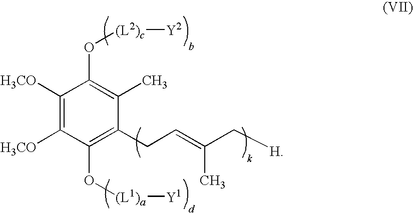

In another exemplary embodiment, one of the phenolic hydroxy groups of the ubiquinol analog is derivatized with a hydrophilic moiety of the invention. Exemplary solubilizing agents have the structure:

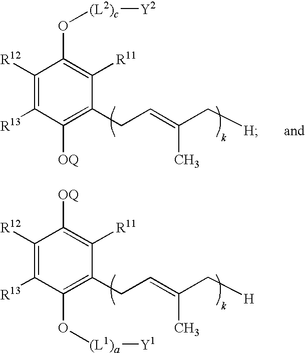

wherein Q is a member selected from H, a negative charge and a salt counter ion.

Exemplary solubilizing agents have a structure according to Formula (VIII),

Formula (IX) or Formula (X):

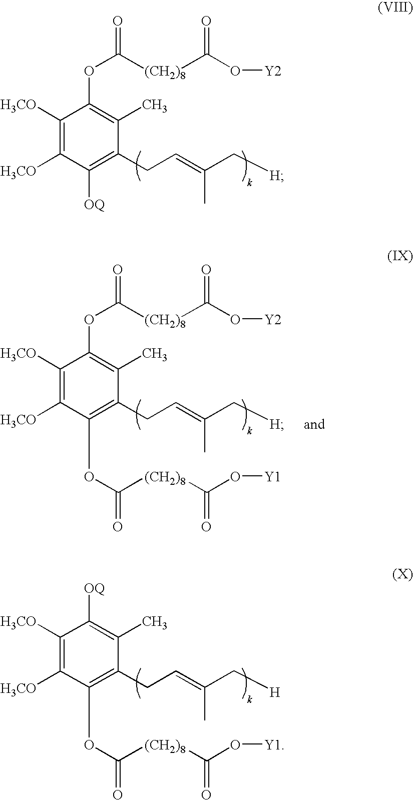

In another exemplary embodiment, one or both of the phenolic hydroxy groups of ubiquinol are part of an ether bond with the linker moiety. Exemplary solubilizing agents have a formula, which is a member selected from:

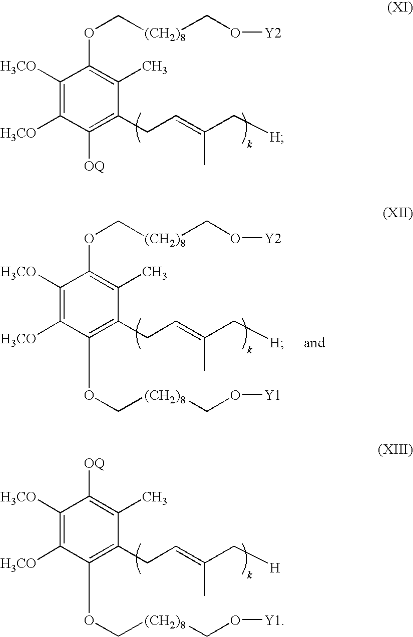

In another exemplary embodiment the invention, the solubilizing agent is a mixture of two or more solubilizing agents described herein. In an exemplary embodiment, the solubilizing agents have a structure according to Formula (V). In one example, the integer k is constant, but at least one of the solubilizing agents includs one hydrophilic moiety, while another includes two hydrophilic moieties. In another embodiment, the mixture includes two regioisomers.

In an exemplary embodiment, the compounds in the mixture of solubilizing agents have structures according to Formulae (VII), (VIII), (IX), (X), (XI), (XII) and (VIII).

Methods of making the above solubilizing agents are known in the art. For example, the methods of making PCS, PTS, and PSS are disclosed in U.S. Pat. Nos. 6,045,826, 6,191,172, 6,632,443, and WO 96/17626, all herein incorporated by reference. The method of making PQS is disclosed in U.S. Patent Application No. 60/915,061, herein incorporated by reference.

### Specific Sterols and Linkers

In an exemplary embodiment, the solubilizing agent has a structure, which is a member selected from:

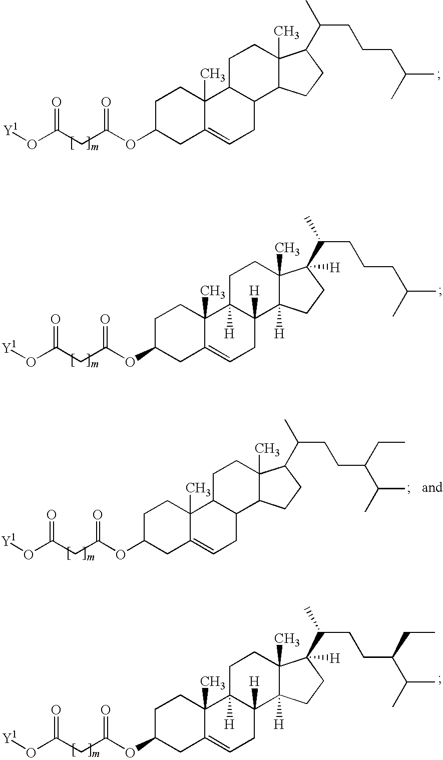

wherein m is a member selected from 2-16 and Y¹ is a hydrophilic moiety. In one example, m is a member selected from 2, 6, 8, 10, 12 and 14. In another example, m is 2. In yet another example, m is 8.

### Specific Sterols and PEG

In an exemplary embodiment, the solubilizing agent is a member selected from

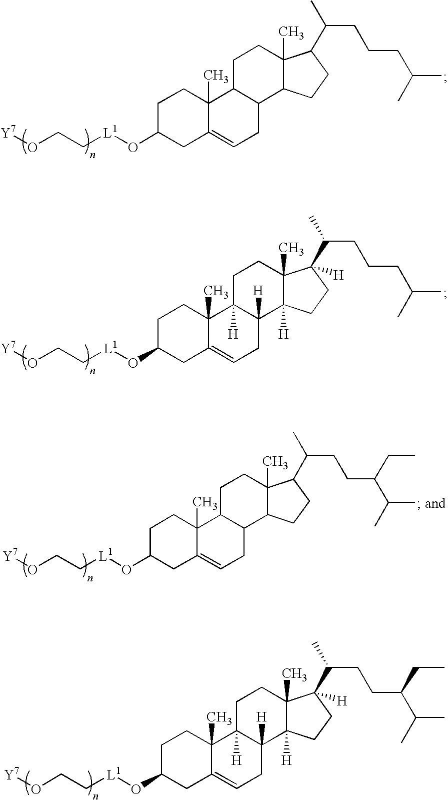

wherein n is a member selected from 10 to 2500, L¹ is a linker moiety, Y⁷ is a member selected from H and methyl.

### Specific Tocopherols and Linkers

In an exemplary embodiment, the solubilizing agent has a structure according to one of the following formulae:

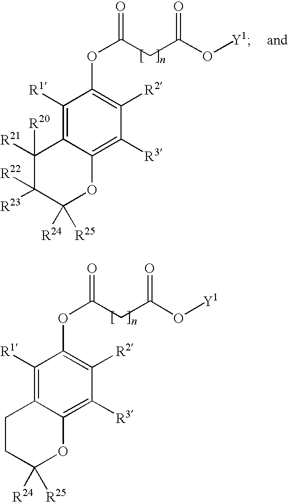

wherein n is an integer selected from 1 to 20. Y¹, R¹, R², R³, R²⁰, R²¹, R²², R²³, R²⁴ and R²⁵ are defined as herein above.

In another exemplary embodiment, R²⁵ includes a moiety having a structure selected from the following formulae:

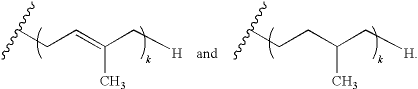

In an exemplary embodiment, k is 3, R³′ is methyl, R²′ is methyl and R¹′ is methyl. In another exemplary embodiment, k is 3, R³′ is methyl, R²′ is H and R¹′ is methyl. In another exemplary embodiment, k is 3, R³′ is methyl, R²′ is methyl and R¹′ is H. In another exemplary embodiment, k is 3, R³′ is methyl, R²′ is H and R¹′ is H.

In another exemplary embodiment, n is 8, k is 3, R³′ is methyl, R²′ is methyl and R¹′ is methyl. In another exemplary embodiment, n is 8, k is 3, R³′ is methyl, R²′ is H and R¹′ is methyl. In another exemplary embodiment, n is 8, k is 3, R³′ is methyl, R²′ is methyl and R¹′ is H. In another exemplary embodiment, n is 8, k is 3, R³′ is methyl, R²′ is H and R¹′ is H.

In another exemplary embodiment, n is 2, k is 3, R³′ is methyl, R²′ is methyl and R¹′ is methyl. In another exemplary embodiment, n is 2, k is 3, R³′ is methyl, R²′ is H and R¹′ is methyl. In another exemplary embodiment, n is 2, k is 3, R³′ is methyl, R²′ is methyl and R¹′ is H. In another exemplary embodiment, n is 2, k is 3, R³′ is methyl, R²′ is H and R¹′ is H.

### Specific Tocopherols and PEG

In an exemplary embodiment, the solubilizing agent has a structure according to the following formula:

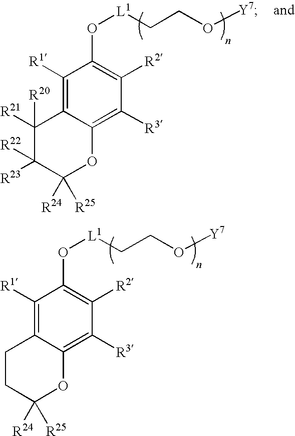

wherein n is a member selected from 10 to 2500. L¹, R¹′, R²′, R³′R²⁰, R²¹, R²², R²³, R²⁴ and R²⁵ are defined as herein above. Y⁷ is selected from H and methyl.

In another exemplary embodiment, R²⁵ includes a moiety having a structure selected from the following formulae:

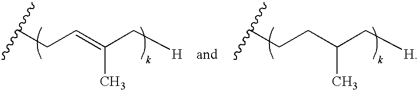

In an exemplary embodiment, k is 3, R³′ is methyl, R²′ is methyl and R¹′ is methyl. In another exemplary embodiment, k is 3, R³′ is methyl, R²′ is H and R¹′ is methyl. In another exemplary embodiment, k is 3, R³′ is methyl, R²′ is methyl and R¹′ is H. In another exemplary embodiment, k is 3, R³′ is methyl, R²′ is H and R¹′ is H.

### Specific Ubiquinols and Linkers

In an exemplary embodiment, the solubilizing agent is a member selected from

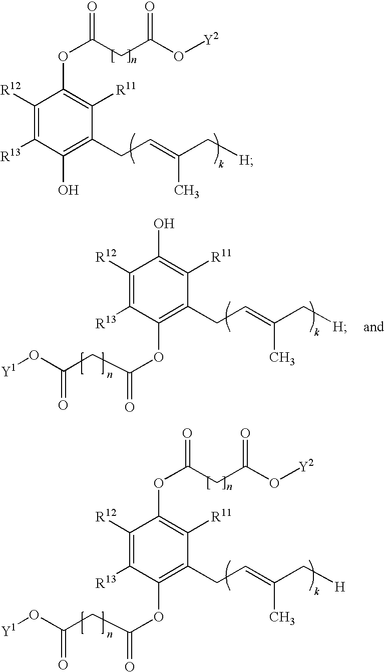

wherein k is a member selected from 1 to 15 and n is a member selected from 1 to 20. Y¹, R¹¹, R¹², and R¹³ defined as herein above. In an exemplary embodiment, k is 10. In another exemplary embodiment, n is 8.

In an exemplary embodiment, the solubilizing agent is a member selected from

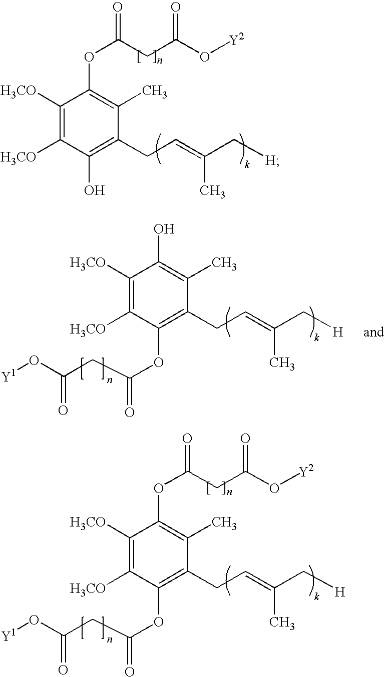

wherein k is a member selected from 1 to 15 and n is a member selected from 1 to 20. Y¹, R¹¹, R¹², R¹³ defined as herein above. In an exemplary embodiment, k is 10. In another exemplary embodiment, n is 8.

### Specific Ubiquinols and PEG

In an exemplary embodiment, the solubilizing agent is a member selected from

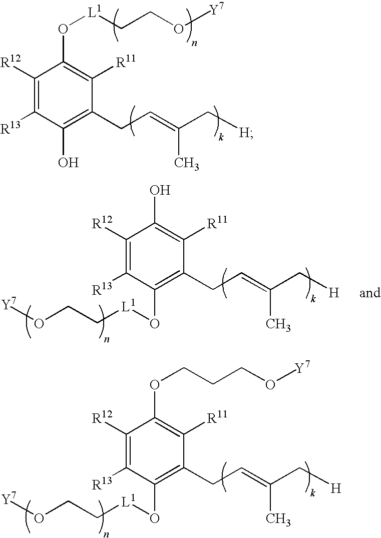

wherein k is a member selected from 1 to 15 and n is a member selected from 10 to 2500. Y¹, R¹¹, R¹², R¹³ are defined as herein above. In an exemplary embodiment, k is 10. Y⁷ is a member selected from H and methyl.

In an exemplary embodiment, the solubilizing agent is a member selected from

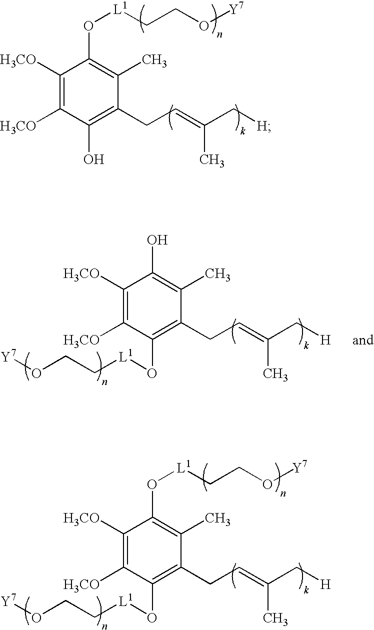

wherein k is a member selected from 1 to 15 and n is a member selected from 10 to 2500. L¹ and Y¹ are defined as herein above. In an exemplary embodiment, k is 10. Y⁷ is a member selected from H and methyl.

In a preferred embodiment, the solubilizing agent is a member selected from polyoxyethanyl-tocopherol-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof.

In an exemplary embodiment, the formulations of the invention include from about 10% to about 50% by weight of a solubilizing agent, such as PTS. Preferably, the formulations include from about 15% to about 40% (w/w) solubilizing agent, more preferably from about 20% to about 40% (w/w), and even more preferably from about 20 to about 35% (w/w).

Solubilizing agents utilized in the compositions of the invention include those described in U.S. Provisional Patent Application 60/773,951; and US Patents: 6,045,826; 6,191,172 and 6,632,443 to Borowy-Borowski et al., which are incorporated herein by reference for all purposes. These solubilizing agents can be purchased commercially from sources such as Zymes (New Jersey) or produced according to the methods described in the above documents.

In an exemplary embodiment, the solubilizing agent contributes to the formation of micelles once the composition is added to an aqueous solution. The size of the formed micelles in solution (particle size) may be measured using a dynamic light scattering (DLS) detector. Typically, smaller particle sizes are associated with a greater tendency of the human body to absorb the active ingredient contained in those particles or micelles. Thus, in one embodiment, the ubiquinone/ubiquinol compositions of the invention form particle sizes in aqueous solution, which are decreased when compared with the particle sizes formed by known formulations. The average particle size formed by the compositions of the invention in aqueous solution is preferably below about 100 nm. In exemplary embodiment, the average particle size is from about 10 nm to about 90 nm. An exemplary average particle size is from about 5 nm to about 70 nm, preferably from about 10 nm to about 50 nm, more preferably from about 10 nm to about 30 nm, and most preferably from about 15 nm to about 25 nm. In one embodiment, the particle size is between about 300 nm and about 1 nm, preferably between about 200 nm and about 1 nm, more preferably between about 100 nm and about 1 nm. Smaller particle sizes are generally preferred.

In an exemplary embodiment, the formulations of the invention include from about 5% to about 50% by weight of a solubilizing agent, such as PQS. Preferably, the formulations include from about 10% to about 30% (w/w) solubilizing agent, more preferably from about 15% to about 25% (w/w).

In another exemplary embodiment, the solubilizing agent is added to the compositions of the invention at a ratio of ubiquinone/ubiquinol to surfactant from about 1:1 to about 1:5 (w/w), preferably from about 1:1 to about 1:3 (w/w), and more preferably from about 1:1 to about 1:2 (w/w). The upper limit of the weight ratio is not critical, and the solubilizing agent can be used in any excess. This is not desirable however, as increasing the amount of the solubilizing agent decreases the concentration of the active ingredient in the composition and in its aqueous solutions.

The water-soluble compositions of the present invention can be prepared by two different procedures, either in the presence or in the absence of an auxiliary organic solvent. In the first case, a lipophilic compound and a solubilizing agent are first dissolved in a predetermined molar ratio in a water-miscible organic solvent and this solution is then diluted with a predetermined amount of water, without precipitation of the lipophilic compound. The organic solvent and water are then removed by evaporation under reduced pressure. A volatile organic solvent is usually removed first, followed by water, in which case the amount of water removed from the solution may be controlled, to achieve a desired concentration of the water-soluble composition in the remaining concentrate. Alternatively, both the organic solvent and water are removed by evaporation, and the waxy residue is reconstituted with a suitable aqueous medium (such as water, physiological saline, or a buffer solution), to provide a clear aqueous solution.

The organic solvent used in the above procedure should be a good solvent for both the lipophilic compound and the solubilizing agent and has to be miscible with water. If a water-soluble composition is to be used in a pharmaceutical formulation, this solvent should be also pharmaceutically acceptable, as the removal of the solvent by evaporation may not always be possible. Examples of solvents suitable for the practice of the invention are tetrahydrofuran, ethanol, ethylene glycol, propylene glycol, and acetic acid. Solvents with a low boiling point, such as tetrahydrofuran, are preferred.

The amount of the organic solvent is not critical, and is equal to or greater than the minimum amount of solvent necessary to dissolve the given amounts of the lipophilic compound and solubilizing agent. The amount of water used for the dilution is also not critical, and is preferably between 10 to 25 times the volume of the organic solvent.

An alternative procedure for preparing water-soluble compositions according to the invention consists of preparing first a mixture of an ubiquinone or ubiquinol compound and a solubilizing agent in a predetermined molar ratio. This mixture is then heated to a temperature higher than the respective melting points of the ubiquinone or ubiquinol and the solubilizing agent, for a time necessary to obtain a clear melt, which process can be seen as dissolution of the ubiquinone or ubiquinol compound in the solubilizing agent. The melt so obtained can be reconstituted with a predetermined amount of a suitable aqueous medium, to provide a clear aqueous solution of a desired concentration. This method of preparing water-soluble compositions of the invention is preferred, as it is simpler and avoids limitations of the procedure that relies on an auxiliary organic solvent, such as the pharmaceutically acceptable character of the solvent required for most applications.

The ability of solubilizing agents of the present invention to dissolve an ubiquinone or ubiquinol compounds in the absence of an auxiliary organic solvent can be used for preparing water-soluble forms of bioactive compounds, in particular coenzyme Q₁₀, without purifying it by crystallization after synthesis, thus reducing the overall cost of preparing water-soluble compositions of this compound.

Many compositions of the present invention show a decreasing solubility in water with increasing temperature of the solution. This provides an alternative method of isolation and/or purification of such compositions. For the purpose of purification, the composition is dissolved in water at a ratio of the composition to water generally not exceeding 1:2 by volume and the solution is heated, for example in a boiling water bath, for a time necessary to achieve the separation of the water-soluble composition as a liquid phase, usually a few minutes. The oily phase is then separated from the hot solution, while keeping the temperature of the solution substantially unchanged, as cooling of the solution would increase the solubility of the composition and result in a reduced yield of the recovery. A speedy separation of the oily phase to avoid the cooling can be achieved, for example, by centrifugation.

The water-soluble compositions of the present invention have a waxy consistence and may be difficult to manipulate in this highly concentrated form. To make them more easily processable as solids, they may be combined with suitable solid, water-soluble additives, such as vitamin C, gelatin, a protein supplement, or a polyethylene glycol. In some embodiments, polyethylene glycol is preferred. In the latter case, a polyethylene glycol having an average molecular weight greater than about 5000 is preferred. The ratio of the composition and the additive is not critical, but will be usually limited to avoid an unnecessary dilution of the active ingredient by the additive.

The compositions of the present invention generally show an excellent solubility in water and allow the preparation of aqueous solutions of almost any concentration. As the concentrated solutions can be diluted with an aqueous medium in any proportion and over a wide range of pH conditions without precipitation of the lipophilic compound, the solubility of the compound is maintained in cell culture medium solutions.

### Methods of Cell Culture

The present invention provides methods for the culturing of primary culture, cell lines, tissues, as well as organs.

The cell could be from a variety of organisms, include, but is not limited to animal, plant, bacteria, and fungus.

The cell-types that can be used in the present invention are derived from various tissues, can be of human origin or that of any other mammal, and may be of any suitable source, such as fibroblast cells, stem cells, ovary cell, or cells from pancreas, parotid gland, thyroid gland, parathyroid gland, prostate gland, lachrymal gland, cartilage, kidney, inner ear, liver, parathyroid gland, oral mucosa, sweat gland, hair follicle, adrenal cortex, urethra, and bladder, or portions or multiples thereof. Particularly the media and methods provided herein can be used to culture neurons and glia, either primary or cell lines

The tissue is prepared using any suitable method, such as by gently teasing apart the excised tissue or by digestion of excised tissue with collagenase via, for example, perfusion through a duct or simple incubation of, for example, teased tissue in a collagenase-containing buffer of suitable pH and tonic strength. The prepared tissue then is concentrated using suitable methods and materials, such as centrifugation through ficol gradients for concentration (and partial purification). The concentrated tissue then is resuspended into any suitable vessel, such as tissue culture glassware or plasticware. The resuspended material may include whole substructures of the tissue, cells and clusters of cells. For example, such substructures may include fibroblast cells.

The initial culture of re-suspended tissue cells is a primary culture. In the initial culturing of the primary culture, the cells attach and spread on the surface of a suitable culture vessel with concomitant cell division. Subsequent to the initial culture, and usually after the realization of a monolayer of cells in the culture vessel, serially propagated secondary and subsequent cultures are prepared by dissociating the cells of the primary culture and diluting the initial culture or its succeeding cultures into fresh culture vessels, a procedure known in the art as passaging. Such passaging results in an expanded culture of cells of the originating tissue. The cell culture is passaged at suitable intervals, such as about once a week or after about two to about three cell divisions of the cultured cells. Longer intervals of two to three weeks or shorter intervals of two to three days would suffice also. For passaging the cell cultures, a dilution of the cultured cells at a ratio of from about 1:2 to about 1:100 is used. Preferably, a ratio of from about 1:4 to about 1:50 is used. More preferably, a ratio of from about 1:4 to about 1:6 is used.

The concentrated prepared tissue, which may be in the form of free cells and/or clumps (where the clumps may constitute ordered substructures of the tissue) is re-suspended at any suitable initial cell or presumptive cell density. Suitable cell densities range from about 100 cells to about 1000 cells per square centimeter of surface area of the culture vessel. For useful vessels, see U.S. Pat. No. 5,274,084 and patents and publications cited therein, all incorporated by reference.

The methods to obtain these cells are known in the art. General methods for cell cultures are known in the art. Culture of Animal Cells: A Manual of Basic Technique, Freshney Wiley-Liss (5th Ed. 2005), herein incorporated by reference in its entirety.

Animal cells which can be cultured in a medium of the present invention are not particularly limited and they can be either established cell lines or nonestablished normal cells obtained from biological tissues. Accordingly, animal cells of the present invention can be, for example, cells which can produce proteins by themselves, cells which are transformed by genetic engineering to express heterologous proteins, or cells which are infected with various virus vectors.

Examples of the cells which can produce proteins by themselves include hybridoma cells producing monoclonal antibodies, leukocytes producing interferon (IFN)-α, fibroblasts producing IFN-β, lymphocytes producing IFN-γ, human kidney cells producing prourokinase (pro-UK) or UK, melanoma cells producing tissue plasminogen activator (tPA), In-111 cells producing insulin, HIT cells producing glucagon, HepG2 cells producing erythropoietin, and B151K12 cells producing interleukin-5

Examples of the cell lines transformed by genetic engineering include Vero cells, HeLa cells, CHO (Chinese hamster ovary) cells, HKG cells, NIH3T3 cells, BHK cells, COS-1 cells, COS-7 cells, and myeloma cells.

Examples of the cells infected with virus vectors include those infected with retrovirus vectors, lentivirus vectors, adenovirus vectors, adeno-associated virus vectors, and herpesvirus vectors. These virus vectors can be genetically recombined by an ordinary genetic engineering method. Further, examples of the animal cells which are infected with these virus vectors and cultured using the medium of the present invention include HEK (human embryonic kidney) 293 cells, A549 cells, and PER.C6 cells.

Another preferred embodiment of the present invention provides a method of culturing animal cells, which comprises the steps of adding the medium supplement of the present invention to an animal cell culture medium and culturing animal cells using the resulting medium to grow the animal cells.

Culture conditions for this method, for example, the oxygen concentration, osmotic pressure, pH, temperature of the medium, can be appropriately changed depending on the kind of the cells to be cultured, the purpose of the culture, the volume of the culture, and the kind of the basal medium composition. Any culture system such as batch culture, continuous culture or perfusion culture can be used. High density culture can also be used.

### Applications

The methods provided herein find uses in a variety of applications. The cells grown using culture media and culture media supplements provided herein can find use in many applications, include but not limited to, assays, testing, cell biology research, DNA production, RNA production, protein production, virus production, vaccine production, drug screening and testing, cell production, tissue engineering, organ transplantation, ex vivo transplantation, and gene therapy.

In some embodiments the present invention provides a method of producing a protein, comprising the steps of adding the medium supplement of the present invention to an animal cell culture medium, culturing animal cells capable of producing the protein using the resulting medium to grow the animal cells, and recovering the produced protein from said medium and/or said animal cells.

In the method of producing a protein according to the present invention, examples of the protein which can preferably be produced include monoclonal antibodies, IFN-α, IFN-β, INF-γ, pro-UK or UK, tPA, insulin, glucagon, erythropoietin, and interleukin-5

The protein produced can be recovered using chemical or physical characteristics of the protein and isolated and purified by various ordinary isolation methods. For example, the protein can be recovered, isolated and purified by treatment with a protein coagulant, ultrafiltration, absorption chromatography, ion-exchange chromatography, affinity chromatography, molecular sieving chromatography, dialysis or the like, singly or in combination.

In some embodiments, the present invention provides a method of replicating a virus vector, which comprises the steps of adding the medium supplement of the present invention to an animal cell culture medium, culturing to grow animal cells infected with the virus vectors using the resulting medium and recovering the produced virus vectors from said medium and/or said animal cells.

Virus vectors replicable by the method of replication of the present invention are various virus vectors described above as examples and can be created by genetic recombination, if necessary. Appropriately selected animal cells are infected with the virus vectors of interest by methods known in the art. Further, the virus vectors can be recovered from grown cells by isolation and purification using various ordinary isolation methods such as ultrafiltration and centrifugation. Here it is desirable to appropriately select the method of recovering virus vectors according to the kind of virus vectors.

Generally, gene therapies are categorized into two kinds, i.e., ex vivo gene therapy and in vivo gene therapy. The former is a therapeutic method in which cells derived from a patient are first cultured outside the body and then treated for gene transfer, after which the cells are administered to the patient. The latter is a therapeutic method in which vectors with transferred genes are directly introduced into the patient's body.

The method according to the present invention can replicate virus vectors, into which genes used for such gene therapies are introduced, more efficiently than conventional methods. Further, the medium of the present invention exhibits excellent growth stimulating effect on the animal cells used for such a replication method, such as 293 cells.

In one aspect, the present invention provides composition and methods for treating patients with cells or components of cells grown in a cell culture media of the invention. The cells grown via the present invention may be any type of cells including stems cells which may be embryonic stem cells or adult stem cells, cells from specific organs including but limited to heart, lung, skin, pancreas and liver. The different cells may be obtained from the same patient as the platelets are used to treat the same patient. A range of different therapeutic results can be obtained. For example, heart tissue regrown, skin grafting enhanced, and diabetic patients treated by growing cells which produce insulin. Accordingly, the term “treatment” is intended to mean providing a therapeutically detectable and beneficial effect of any kind on a patient.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

### Methods of Treatment

The method of the present invention utilizing solubilized ubiquinones and ubiquinols enhances the viability of cells of the nervous system in culture and in vivo. Accordingly, the present invention provides methods of treating diseases of the nervous system, such as peripheral neuropathy. Peripheral neuropathy refers to damage to nerves of the peripheral nervous system. It may be caused either by diseases of the nerve or from the side-effects of systemic illness, most often manifested as one or a combination of motor, sensory, sensorimotor, or autonomic neural dysfunction. The wide variety of morphologies exhibited by peripheral neuropathies can each be uniquely attributed to an equally wide variety of causes. For instance, peripheral neuropathies can be genetically acquired, can result from a systemic disease, or can be induced by a toxic agent. Some toxic agents that cause neurotoxicities are therapeutic drugs, antineoplastic agents, contaminants in foods or medicinals, and environmental and industrial pollutants.

Peripheral neuropathy is the major neurological complication observed among persons infected with human immunodeficiency virus type 1 (HIV-1) in the developed world.

The most common form of peripheral neuropathy is sensory polyneuropathy (HIV_SN), affecting as many as 35% of individuals with acquired immunodeficiency syndrome (AIDS). There are two principal forms of HIV-SN, distal sensory polyneuropathy (DSP) and antiretroviral-induced toxic neuropathy (ATN). In both types of sensory neuropathy, features are defined by pain, paresthesiae, gait instability, and autonomic dysfunction. HIV-SN is characterized by prominent small-diameter axonal loss, following a “dying back” m pattern of degeneration, likely driven by local inflammation within the nerve and dorsal root ganglia (DRGs) with ensuing neuronal injury. The development of ATN has been associated with the use of the nucleoside analogue reverse transcriptase inhibitors (NRTI) didanosine (ddI), zalcitabine (ddC), and stavudine (d4T). ATN is indistinguishable from DSP clinical examination except for the history of recent NRTI use. The small-diameter nociceptive sensory axons and their respective soma in the DRGs are the principal cellular structures affected in HIV-induced DSP or NRTI-induced ATN. However, ATN may be a synergistic consequence of HIV infection together with the neurotoxic effects of select antiretroviral drugs, because some HIV-uninfected animals, treated with neurotoxic antiretroviral drugs, do not develop ATN-like phenotype. Although select NRTIs are associated with ATN, multiple antiretroviral regimens including those with protease inhibitors (Pls) appear to have adverse effects on metabolism with increased risks for mitochondrial toxicity, hyperglycemia, hyperlipidemia, and the lipodystrophy syndrome. Pettersen et al., Ann Neurol 59:816-824 (2006).

Treatment of peripheral neuropathy with acetyl-L-carnitine (ALCAR) has shown short-term as well as long-term symptomatic and histological improvement. Hart, AIDS 18(11):1549-60 (2004); Herzmann et al., HIV Clin Trials. 6(6):344-50 (2005); Osio M., et al., J Peripher Nery Syst. 11(1):72-6 (2006).

N-4-carboxaphenyl-3-(6-oxohydropurin-9-yl)propananide AIT-082 has been disclosed for treatment of drug-induced peripheral neuropathy, particularly drug-induced peripheral neuropathy associated with the administration of oncolytic drugs. U.S. Pat. No. 6,630,478. It also had been proposed to use insulin-like growth factor-I (IGF-I) and insulin-like growth factor-111 (IGF-III) to treat peripheral neuropathy, such as neuropathies associated with systematic disease, genetically acquired neuropathies, and neuropathies caused by a toxic agent. U.S. Pat. Nos. 5,420,112 and 5,633,228.

The present invention provides methods for the treatment, reduction or amelioration of peripheral neuropathy, including antiretroviral toxic neuropathy (ATN)—a common and typically irreversible complication of stavudine (d4T) and didanosine (ddI). The method includes administering to a subject in need of such treatment, an effective amount of a solubilized ubiquinone or ubiquinol according to the disclosure set forth in relation to any of the embodiments above.

Peripheral neuropathy is a condition caused by damage to the peripheral nervous system. Thus, protection of nerve tissues from damages should be able to ameliorate or cure peripheral neuropathy. Coenzyme Q₁₀ is one of the compounds that have been found to have such protection effects.

Coenzyme Q₁₀ has been found to have a neuroprotective effect against glutamate toxicity, and was suggested to be used in treatment of neurological diseases associated with glutamate excitotoxcity, Sandhu et al., Journal of Neuroscience Research, 72:691-703 (2003). Coenzyme Q₁₀ has also been proposed to be used in treatment of neurodegenerative diseases. Matthews et al., P.N.A.S. 95:8892-8897 (1998); McCarthy et al., Toxicology and Applied Pharmacology, 201:21-23 (2004); and Somayajulu et al., Neurobiology of Disease, 18:618-627 (2005).

There are both animal models and cell culture models for peripheral neuropathy. For example, rat model of HIV-associated sensory neuropathy has been established. Keswani et al., The Journal of Neuroscience, 26(40): 10299-10304 (2006). Rat dorsal root ganglia (DRG) cell culture is a wildly used model for studying peripheral neuropathy and its therapy. See e.g. Keswani, Ann Neurol 54:287-296 (2003); Pettersen, et al., Ann Neurol 59:816-824 (2006); and U.S. Pat. No. 6,630,478. Dorsal root ganglion (or spinal ganglion) is a nodule on a dorsal root that contains cell bodies of neurons in afferent spinal nerves. All of the axons in the dorsal root convey somatosensory information, bringing sensory information into the brain and spinal cord. These neurons are of the pseudo-unipolar type, meaning they have two axons, one that conveys sensory information from the body to the soma of the neuron and one from the soma to the junction in the dorsal horn of the spinal cord.

In one aspect, the present invention provides method and composition comprising ubiquinone or ubiquinol (such as coenzyme Q, especially coenzyme Q₁₀) for the prevention and treatment of neurological disorders, particularly peripheral neuropathy, such as neuropathies associated with systematic disease, genetically acquired neuropathies, and neuropathies caused by a toxic agent. Toxic agents include, but not limited to agents used in chemical therapy for cancer or AIDS, such as stavudine (d4T) and didanosine (ddI).

In another aspect, the present invention provides a method for protecting neurons in a mammalian subject against anti-viral toxic agent, comprising administering to a subject in need of protecting a therapeutically effective amount of a pharmaceutical composition provided herein.

In one aspect, the present invention provides method and composition comprising coenzyme Q (such as coenzyme Q₁₀) for the prevention of antiretroviral toxic neuropathy (ATN)—a common and typically irreversible complication of stavudine (d4T) and didanosine (ddI). The development of a novel water-soluble formulation of coenzyme Q₁₀ (Ubisol-Aqua™:Coenzyme Q₁₀-denoted here as H_{Q}O™) has facilitated testing of this strategy in a laboratory model of ATN, which is described in more detail in the examples.

The methods of the invention provide for administration of a composition, described supra, with a dose, dosing schedule and dosing duration sufficient to treat, prevent or ameliorate peripheral neuropathy or symptoms thereof. In an exemplary embodiment, the dose of coenzyme Q can be about 0.1 mg to about 4,000 mg. In another exemplary embodiment, the dose of Coenzyme Q can be about 20 mg, about 50 mg, about 100 mg, about 200 mg, about 500 mg, about 1,000 mg, about 1,200 mg, about 1,500 mg or about 2,000 mg.

The methods of the invention provide for administration of a composition on a dosing schedule that provides a therapeutic or prophylactic amount of the composition. In an exemplary embodiment, the composition can be administered about four times daily, about three times daily, about twice daily, about daily, about every other day, about three times weekly, about twice weekly, about weekly or about every two weeks.

The methods of the invention provide that the composition can be administered for a duration sufficient to provide a therapeutic or prophylactic effect. In an exemplary embodiment, the composition can be administered, for example, daily for one year. In another embodiment, the composition can be administered for about six months, about a year, about two years, about five years, about 10 years or indefinitely. In a particularly preferred embodiment, the composition can be administered daily for five years.

It will be apparent to those of skill in the art that the dose, dosing schedule and duration can be adjusted for the needs of the particular subject taking into consideration the subject's age, weight, severity of disease and other co-morbid conditions.

In another aspect, the composition and method provided herein can be used in combination with other compositions and methods that can be used to ameliorate or cure peripheral neuropathy.

For example, although there is no proven treatment available for HIV-related neuropathy, there are several options available for managing the symptoms.

Gabapentin (Neurontin), an anti-seizure medication, has been studied and proven effective in diabetic neuropathy, which is thought to be similar to HIV-related neuropathy. For night-time pain relief, Neurontin can be combined with topical pain relievers such as Lidocaine ointment and the Lidoderm patch. Capsaicin, available over the counter as Capzasin-P and Capzasin-HP, is another option for topical pain relief.

Other anti-seizure medications such as carbamazepine (Tegretol) and phenyloin (Dilantin) are useful for treating symptoms of neuropathy, but they can lower the amount of HIV-fighting medication in the body. Lamotrigine (Lamictal), another anti-seizure medication, works differently from carbamazepine and phenyloin to reduce symptoms.

In another aspect, the present invention provides composition a ubiquinone or ubiquinol and a solubilizing agent provided herein and a second compound that can be used to ameliorate or cure peripheral neuropathy, include but not limited to: Neurontin, Capsaicin, carbamazepine, phenyloin, and lamotrigine.

In an exemplary embodiment, the invention provides a method of reducing neurotoxicity through administering a therapeutically effective amount of a composition/formulation described herein to a mammal.

In an exemplary embodiment, the invention provides a method of treating peripheral neuropathy through administering a therapeutically effective amount of a composition/formulation described herein to a mammal.

Exemplary embodiments are summarized herein below.

In an exemplary embodiment, the invention provides a water-soluble cell culture medium supplement composition, which includes a ubiquinone or ubiquinol; and a solubilizing agent having the structure according to the following formula:

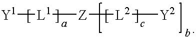

In this formula, the index a is an integer selected from 0 and 1; the index b is an integer selected from 0 and 1; and the index c is an integer selected from 0 and 1. Z is ubiquinol. Y¹ and Y² are hydrophilic moieties which are members selected from polyethers, polyalcohols and derivatives thereof. L¹ and L² are linkers.

In an exemplary embodiment, according to the above paragraph, the ubiquinone or ubiquinol has a structure which is a member selected from Formula (I) and Formula (II):

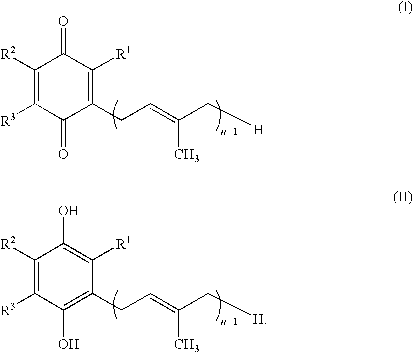

In these formulas, integer n is a member selected from 0 to 13. R¹, R² and R³ are members independently selected from H, substituted or unsubstituted alkyl and substituted or unsubstituted alkoxy.

In an exemplary embodiment, according to any of the above paragraphs, the ubiquinone or ubiquinol is Coenzyme Q₁₀.

In an exemplary embodiment, according to any of the above paragraphs, the ubiquinone or ubiquinol is a Coenzyme Q₁₀ analog.

In an exemplary embodiment, according to any of the above paragraphs, the ubiquinone or ubiquinol is Coenzyme Q₁₀ and said solubilizing agent is PQS.

In an exemplary embodiment, the invention provides a culture medium composition, comprising: a) a basal culture medium; and b) a water-soluble cell culture medium supplement composition described herein.

In an exemplary embodiment, according to any of the above paragraphs, the basal medium is selected from the group consisting of RPMI 1640, Dulbecco's modified Eagle's Medium, and Ham's F12.

In an exemplary embodiment, the invention provides a culture medium solution comprising: a) a basal culture medium; and b) water-soluble cell culture medium supplement composition described herein, wherein the medium and the supplement composition are dissolved in water.

In an exemplary embodiment, according to any of the above paragraphs, the basal medium is selected from the group consisting of RPMI 1640, Dulbecco's modified Eagle's Medium, and Ham's F12.

In an exemplary embodiment, according to any of the above paragraphs, the solution further comprises serum.

In an exemplary embodiment, according to any of the above paragraphs, the serum is fetal calf serum.

In an exemplary embodiment, according to any of the above paragraphs, the solution further comprises a growth factor.

In an exemplary embodiment, according to any of the above paragraphs, the growth factor is nerve growth factor.

In an exemplary embodiment, the invention provides a method of growing a cell. This method comprises the step of contacting said cells with a cell culture medium comprising: a) a basal culture medium; b) a ubiquinone or ubiquinol; and c) a solubilizing agent having the structure according to the following formula:

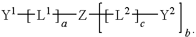

In this formula, the index a is an integer selected from 0 and 1; the index b is an integer selected from 0 and 1; and the index c is an integer selected from 0 and 1. Z is ubiquinol. Y¹ and Y² are hydrophilic moieties which are members selected from polyethers, polyalcohols and derivatives thereof; and L¹ and L² are linkers.

In an exemplary embodiment, according to any of the above paragraphs, the ubiquinone or ubiquinol having has a structure which is a member selected from Formula (I) and Formula (II):

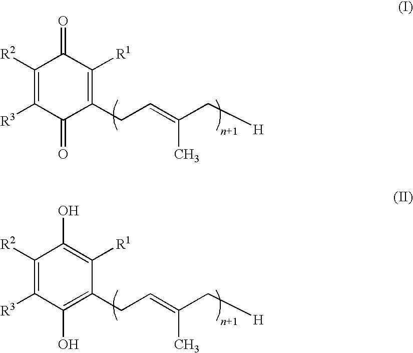

wherein the integer n is a member selected from 0 to 13, R¹, R² and R³ are members independently selected from H, substituted or unsubstituted alkyl and substituted or unsubstituted alkoxy.

In an exemplary embodiment, according to any of the above paragraphs, the ubiquinone or ubiquinol is Coenzyme Q₁₀.

In an exemplary embodiment, according to any of the above paragraphs, the ubiquinone or ubiquinol is a Coenzyme Q₁₀ analog.

In an exemplary embodiment, according to any of the above paragraphs, the ubiquinone or ubiquinol is Coenzyme Q₁₀ and said solubilizing agent is PQS.

In an exemplary embodiment, according to any of the above paragraphs, the cell is a neuron.

In an exemplary embodiment, according to any of the above paragraphs, the cell is a glia.

In an exemplary embodiment, according to any of the above paragraphs, the glia is a Schwann cell.

In an exemplary embodiment, according to any of the above paragraphs, the cell is a primary cell culture cell.

In an exemplary embodiment, according to any of the above paragraphs, the cell is derived from a cell line.

In an exemplary embodiment, the invention provides a method of culturing tissue or an organ, said method comprising the step of contacting said tissue with a tissue culture medium comprising: a) a basal culture medium; b) a ubiquinone or ubiquinol; and c) a solubilizing agent having the structure according to the following formula:

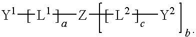

In this formula, the index a is an integer selected from 0 and 1; the index b is an integer selected from 0 and 1; and the index c is an integer selected from 0 and 1. Z is ubiquinol. Y¹ and Y² are hydrophilic moieties which are members selected from polyethers, polyalcohols and derivatives thereof; and L¹ and L² are linkers.

In an exemplary embodiment, according to any of the above paragraphs, the ubiquinone or ubiquinol has a structure which is a member selected from Formula (I) and Formula (II):

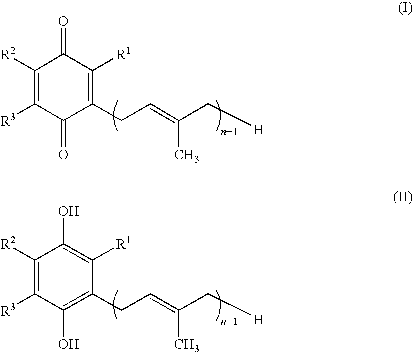

wherein the integer n is a member selected from 0 to 13, R¹, R² and R³ are members independently selected from H, substituted or unsubstituted alkyl and substituted or unsubstituted alkoxy.

In an exemplary embodiment, according to any of the above paragraphs, the ubiquinone or ubiquinol is Coenzyme Q₁₀.

In an exemplary embodiment, according to any of the above paragraphs, the ubiquinone or ubiquinol is a Coenzyme Q₁₀ analog.

In an exemplary embodiment, according to any of the above paragraphs, the ubiquinone or ubiquinol is Coenzyme Q₁₀ and said solubilizing agent is PQS.

Exemplary aspects of the present invention are illustrated by the non-limiting examples below.

### EXAMPLES

### Example 1

Coenzyme 010 Dose Response in Fetal Rat DRGs

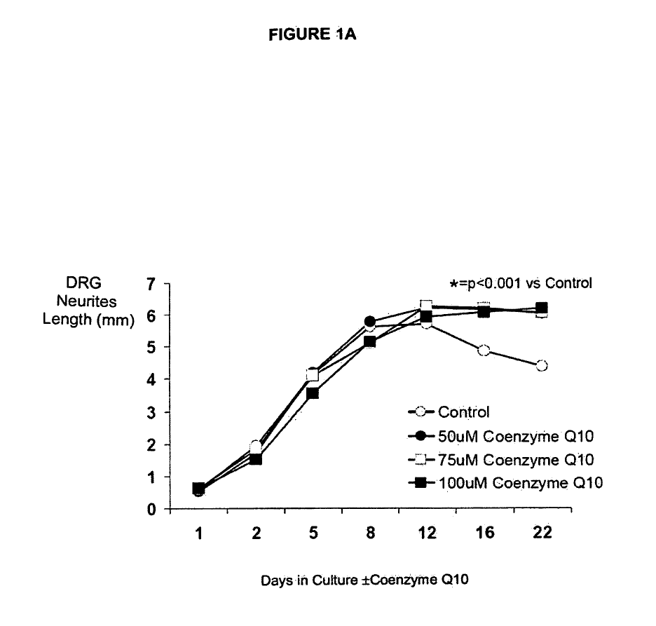

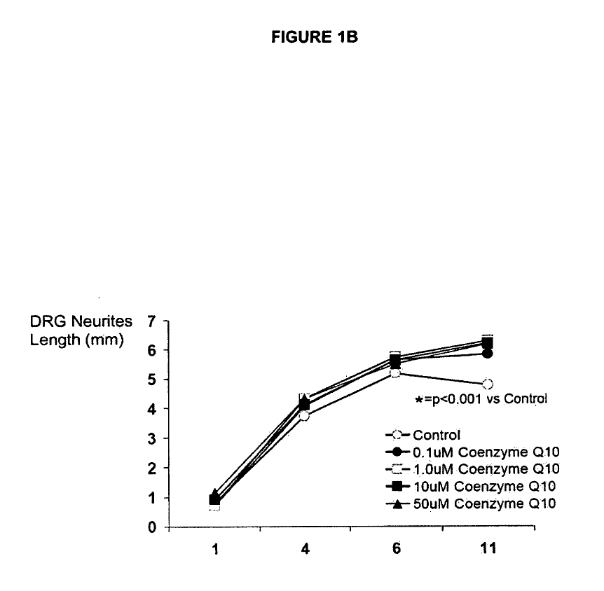

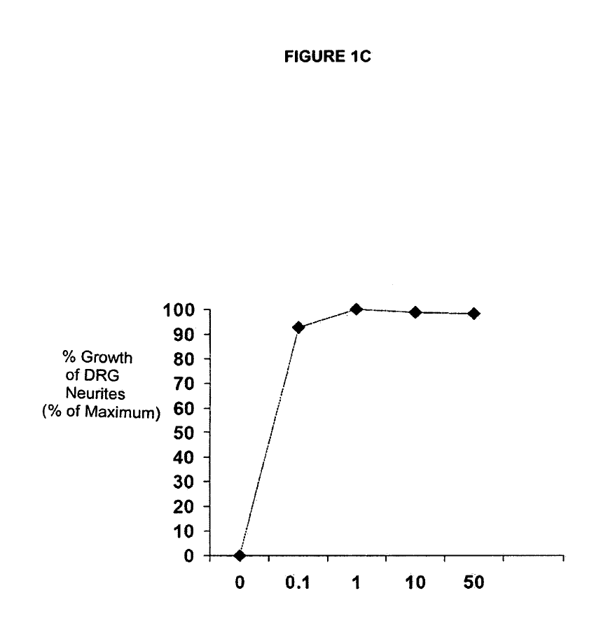

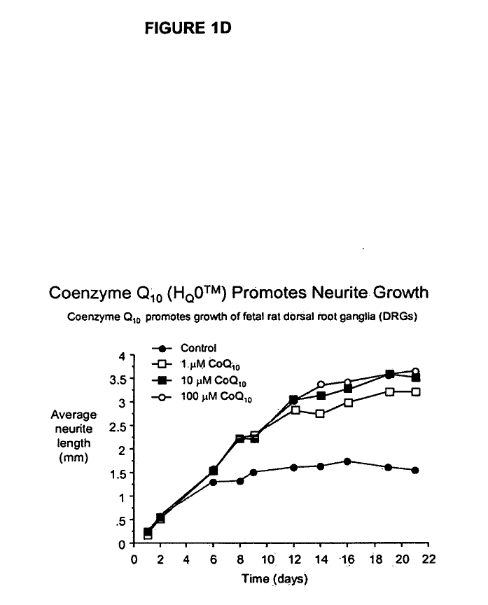

The first CoQ10 dose finding experiment found that there was no evidence of toxicity in cultured fetal rat DRGs exposed to concentrations of CoQ10 of up to 50 uM. In fact, by day 11, the length of neurites around DRGs was longer if the DRGs were cultured with CoQ10 (concentrations ranged from 0.1-50 uM) compared with control DRGs, p<0.0001 for all. FIG. 1A, 1B, 1C.

The aim of the second experiment was to confirm that exposure to 50 uM CoQ10 is not toxic to cultured fetal rat DRGs and also to see if even higher concentrations may be used safely. Eight DRGs (2 plates of 4 DRGs each) were cultured under each of the following conditions:
Control
50 uM CoQ10
75 uM CoQ10
100 uM CoQ10

This experiment was carried on for 22 days, which is about as long as DRGs can be sustained in this model, even under control conditions. The experiment was continued for this time period to confirm that CoQ10 remains non-toxic in this model over time.

Neurite lengths around each DRG were measured at four representative points (giving a total of 32 measures at each exposure concentration at each time point) on days 1, 2, 5, 8, 12, 16 and 22.

The pattern seen is very similar to that seen with the first rat. Basically no evidence of toxicity associated with CoQ10 is seen in any of the concentrations assessed. Over time, control DRGs begin to grow less well (presumably due to having exhausted nutrients in the available culture media) and at this point it becomes evident that CoQ10 is associated with improved survival/neurite growth. In the first rat, DRGs exposed to CoQ10 exhibited enhanced neurite growth/survival relative to controls at day 11.

At day 16, DRGs exposed to CoQ10 are clearly more viable as measured by neurite lengths compared with DRGs cultured under control conditions. Unpaired t tests comparing neurite lengths under the different culture conditions are as follows:

50 uM CoQ10 versus controls, mean neurite length P5.15 versus 4.84 mm, p<0.0001

75 uM CoQ10 (non-viable DRG excluded) versus controls, 6.20 versus 4.84 mm, p<0.0001

100 uM CoQ10 versus controls, 6.07 versus 4.84 mm, p<0.0001.

A third experiment measured growth of DRG's with varying doses of H_{Q}O™, 0.1, 10, 100 uM CoQ10. This experiment was done in DRG's isolated from a new rat, which accounts for the difference in neurite length compared to experiments 1 and 2. The protocol is identical to the first experiments and the data confirm the initial findings further demonstrating that H_{Q}O™enhances neurite growth in DRGs. (FIG. 1E)

The data are shown in FIG. 1. FIG. 1A shows enhanced growth in DRGs after treatment of up to 50 μM. FIG. 1B illustrates that the growth of the DRGs is similar after different doses of H_{Q}O™. After 11 days of H_{Q}O™treatment, neurite length of H_{Q}O™ treated DRGs is greater than controls as shown in FIG. 1C. FIG. 1D shows data from the third set of experiments, confirming enhanced growth of DRG's with 0.1, 10, 100 uM H_{Q}O™ beginning the 8^{th} day of treatment. By day 22, the growth is double that of the controls.

### Example 2

Safety and Preventative Efficacy of H_{Q}O™

Objective: To examine the safety and preventative efficacy of H_{Q}O™ using cultured fetal rat dorsal root ganglia (DRGs) as an in vitro model of ATN.

Methods: Fetal rat DRGs were cultured on media containing collagen, nerve growth factor and a range of concentrations of H_{Q}O™ (PTS:CoQ in saline) and/or d4T/ddI (both are 33 μM Neurite outgrowth and DRG survival were monitored using video image analysis.

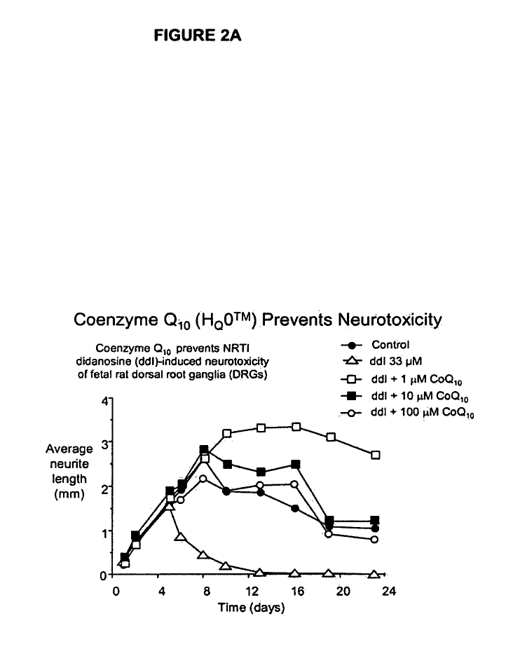

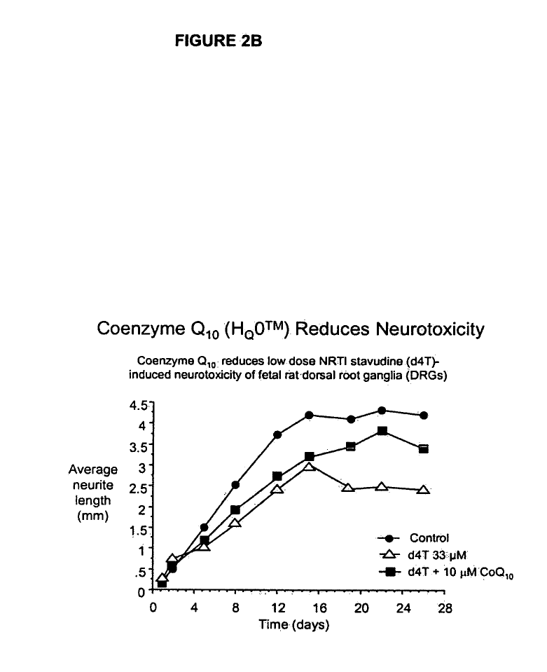

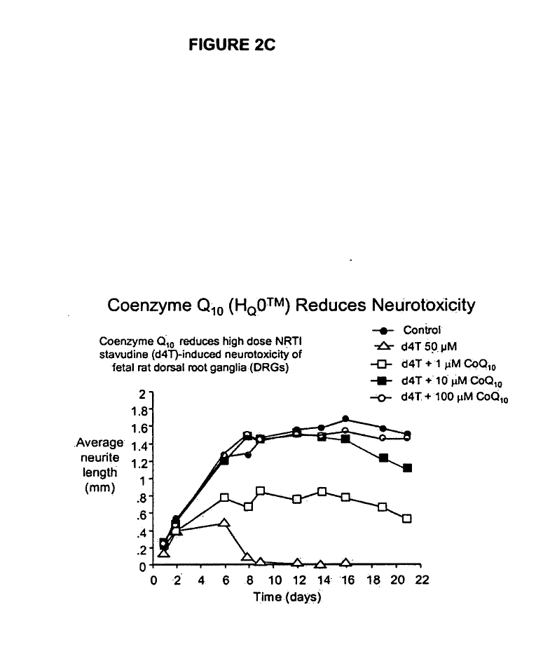

Results: 0.1-100 μM H_{Q}O™ improved neurite growth (p<0.0001 versus controls by day 16) and DRG survival (DRGs cultured with H_{Q}O™remained viable for at least one week beyond controls). H_{Q}O™also reduced the toxicity of d4T and ddI in this model. By day 19 DRGs exposed to 10 μM H_{Q}O™together with 33 μM d4T demonstrated greater neurite growth than those with d4T alone (p=0.01) and were not different from controls. Similarly, 10 μM H_{Q}O™reduced the toxicity of ddI. By day 6 of culture, DRGs exposed to 33 μM ddI exhibited impaired neurite growth compared with controls (p<0.0001). However, DRGs exposed to 1-100 μM H_{Q}O™together with 33 μM ddI showed greater neurite outgrowth compared to those exposed to ddI alone (p<0.0001 for all) and were growing at least as well as control DRGs. (FIGS. 2A and 2B).

In a separate experiment, DRGs were exposed to a higher dose (50 μM) of d4T alone or with 10 or 100 nM H_{Q}O™. By day 8 the toxicity observed in DRGs treated with d4T alone was evident and a clear reversal of toxicity was observed with both 10 and 100 nM

H_{Q}O™. In DRGs treated with H_{Q}O™neurite outgrowth returned to control levels. (FIG. 2C).

Conclusions: H_{Q}O™enhances the growth and survival of cultured fetal rat DRGs, and reduces the toxicity of d4T and ddI in this model. These data suggest that H_{Q}O™ may effectively prevent ATN. Controlled trials are needed to confirm this in a clinical setting, and to examine the role of H_{Q}O™ in treating established ATN.

### Example 3

LAC Prevents NRTI (ddI)-Induced Neurotoxicity of Fetal Rat Dorsal Root Ganglia (DRGs)

Nucleoside analogue Reverse Transcriptase Inhibitors (NRTIs) are potent inhibitors of HIV replication and central to effective Highly Active Anti-Retroviral Therapy (HAART). However, this class of drugs causes mitochondrial toxicity leading to serious end organ damage including neuropathy, lipodystrophy, and metabolic disturbances. Co-administration of micronutrients that improve mitochondrial function such as coenzyme Q10 and L-acetyl carnitine (LAC) reduce the mitochondrial toxicity of NRTIs. LAC originally studied in a validated model of neurotoxicity due to unavailable source of water soluble coenzyme Q10.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A water-soluble cell culture medium supplement composition, comprising:
(1) a ubiquinone or ubiquinol; and
(2) a solubilizing agent having a structure according to the following formula: 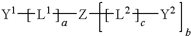
wherein
a is an integer selected from 0 and 1;
b is an integer selected from 0 and 1;
c is an integer selected from 0 and 1;
Z is ubiquinol;
Y¹ and Y² are hydrophilic moieties which are members selected from polyethers, polyalcohols and derivatives thereof; and
L¹ and L² are linkers.

2. The composition according to claim 1, wherein said ubiquinone or ubiquinol has a structure which is a member selected from Formula (I) and Formula (II): 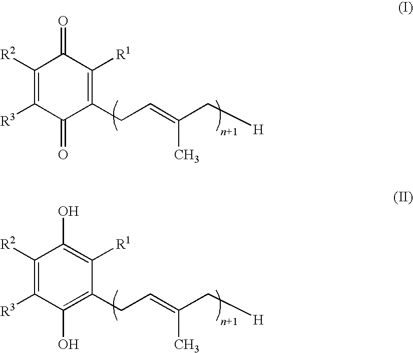
wherein the integer n is a member selected from 0 to 13, R₁, R² and R³ are members independently selected from H, substituted or unsubstituted alkyl and substituted or unsubstituted alkoxy.

3. The composition according to claim 2, wherein said ubiquinone or ubiquinol is Coenzyme Q₁₀.

4. The composition according to claim 2, wherein said ubiquinone or ubiquinol is a Coenzyme Q₁₀ analog.

5. The composition according to claim 2, wherein said ubiquinone or ubiquinol is Coenzyme Q₁₀ and said solubilizing agent is PQS.

6. A culture medium composition, comprising:
a) a basal culture medium; and
b) water-soluble cell culture medium supplement composition according to claim 1.

7. The composition according to claim 6, wherein said basal medium is selected from the group consisting of RPMI 1640, Dulbecco's modified Eagle's Medium, and Ham's F12.

8. A culture medium solution comprising:
a) a basal culture medium; and
b) water-soluble cell culture medium supplement composition according to claim 1, wherein said medium and said supplement composition are dissolved in water.

9. The solution according to claim 8, wherein said basal medium is selected from the group consisting of RPMI 1640, Dulbecco's modified Eagle's Medium, and Ham's F12.

10. The solution according to claim 9, further comprises serum.

11. The solution according to claim 10, wherein said serum is fetal calf serum.

12. The solution according to claim 11, further comprises a growth factor.

13. The solution according to claim 12, wherein said growth factor is nerve growth factor.

14. A method of growing a cell, said method comprising the step of contacting said cells with a cell culture medium comprising:
a) a basal culture medium;
b) a ubiquinone or ubiquinol; and
c) a solubilizing agent having the structure according to the following formula: 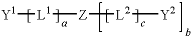
wherein
a is an integer selected from 0 and 1;
b is an integer selected from 0 and 1;
c is an integer selected from 0 and 1;
Z is ubiquinol; Y¹ and Y² are hydrophilic moieties which are members selected from polyethers, polyalcohols and derivatives thereof; and
L¹ and L² are linkers.

15. The method according to claim 14, wherein said ubiquinone or ubiquinol has a structure which is a member selected from Formula (I) and Formula (II): 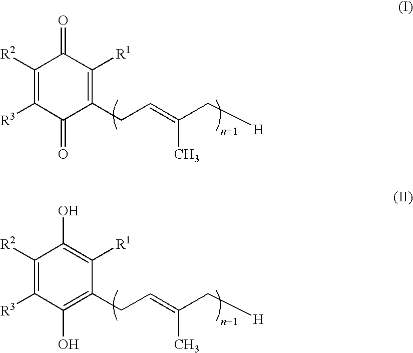
wherein the integer n is a member selected from 0 to 13, R₁, R² and R³ are members independently selected from H, substituted or unsubstituted alkyl and substituted or unsubstituted alkoxy.

16. The composition according to claim 14, wherein said ubiquinone or ubiquinol is Coenzyme Q₁₀.

17. The composition according to claim 14, wherein said ubiquinone or ubiquinol is a Coenzyme Q₁₀ analog.

18. The composition according to claim 14, wherein said ubiquinone or ubiquinol is Coenzyme Q₁₀ and said solubilizing agent is PQS.

19. The method according to claim 14, wherein said cell is a neuron.

20. The method according to claim 14, wherein said cell is a glia.

21. The method according to claim 20, wherein said glia is a Schwann cell.

22. The method according to claim 14, wherein said cell is a primary cell culture cell.

23. The method according to claim 14, wherein said cell is derived from a cell line.

24. A method of culturing tissue or organ, said method comprising the step of contacting said tissue with a tissue culture medium comprising:
a) a basal culture medium;
b) a ubiquinone or ubiquinol; and
c) a solubilizing agent having the structure according to the following formula: 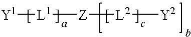
wherein
a is an integer selected from 0 and 1;
b is an integer selected from 0 and 1;
c is an integer selected from 0 and 1;
Z is ubiquinol;
Y¹ and Y² are hydrophilic moieties which are members selected from polyethers, polyalcohols and derivatives thereof; and
L^{l} and L² are linkers.

25. The method according to claim 24, wherein said ubiquinone or ubiquinol has a structure which is a member selected from Formula (I) and Formula (II): 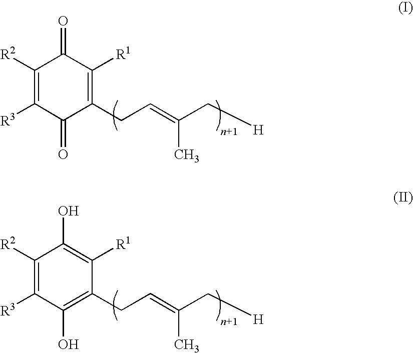
wherein the integer n is a member selected from 0 to 13, R¹, R² and R³ are members independently selected from H, substituted or unsubstituted alkyl and substituted or unsubstituted alkoxy.

26. The composition according to claim 24, wherein said ubiquinone or ubiquinol is Coenzyme Q₁₀.

27. The composition according to claim 24, wherein said ubiquinone or ubiquinol is a Coenzyme Q₁₀ analog.

28. The composition according to claim 24, wherein said ubiquinone or ubiquinol is Coenzyme Q₁₀ and said solubilizing agent is PQS.
